(19) United States
(12) Patent Application Publication
(10) Pub. No.: US 2026/0218133 A1
(43) Pub. Date: Jul. 30, 2026

(54) NANOFIBROUS SCAFFOLD FOR INDUSTRIAL SCALE CELL CULTURING

(71) Applicant: Cellevate AB, Lund (SE)

(72) Inventors: Albin Jakobsson, Lund (SE); Maximilian Ottosson, Lund (SE); Henrik Bratt, Lund (SE); Ulrik Flensborn, Lund (SE); Hira Kausar, Lund (SE)

(21) Appl. No.: 19/140,979

(22) PCT Filed: Dec. 21, 2023
(86) PCT No.: PCT/EP2023/087200

§ 371 Date: Jun. 18, 2025

(30) Foreign Application Priority Data
Dec. 21, 2022 (SE) 2251521-7

(51) Int. Cl. C12N 5/071; C08B 15/10; C12M 1/12

(52) U.S. Cl. CPC C12N 5/0686; C08B 15/10; C12M 25/14; C12N 2531/00; C12N 2533/78; C12N 2537/10; C12N 2539/00

(57) ABSTRACT

The present invention relates to a method for preparing a nanofibrous scaffold and use of the same for promoting cell culturing. In particular, the nanofibrous scaffold comprises crosslinked cellulose nanofibers which yield a continuous microcarrier material for use in industrial scale cell culturing setups.

### TECHNICAL FIELD OF THE INVENTION

The present invention relates to a method for preparing a nanofibrous scaffold and use of the same for promoting cell culturing. In particular, the nanofibrous scaffold comprises crosslinked cellulose nanofibers which yield a continuous microcarrier material for use in industrial scale cell culturing setups.

### BACKGROUND OF THE INVENTION

Biological products (biologics), such as therapeutic proteins, vaccines, or cell and gene therapy products represent a new medical paradigm. Those therapies can provide remarkable outcomes for patients and have already revolutionized the treatment of many diseases in a variety of fields. The most complex biological products with specific glycosylation patterns may be produced by recombinant DNA technology in mammalian cell cultures and includes enzymes, synthetic hormones and monoclonal antibodies. However, manufacturing of biologics is challenging because production of end-products is reliant on sensitive live host cells which demands much more attention than e.g. chemical synthesis of small-molecule drugs.

The biologics manufacturing process can be separated into upstream processes and downstream processes. The upstream process is defined as the entire process from early cell isolation and cultivation to cell banking and culture expansion of the cells until final harvest—in other words the termination of the culture and collection of the live cell batch for purification.

While culturing of cells is therefore the crux of the upstream process, cell immunogenicity, adverse events, and efficacy can all be affected by even the slightest manufacturing process change. As such, it is very difficult to scale up from research to clinical quantities and many promising therapies can not reach the market. In addition, the most significant drawback is low product yield, followed by the costs related to the extensive cell-line development and limited cell viability for production. In all, the manufacturing process is extremely expensive and biologics are not accessible to all patients.

Many cells and tissues, especially mammalian ones, require a surface or other structural support in order to proliferate. To assist the upstream process, microcarriers are therefore routinely used to support growth of adherent cell populations in bioreactors. Microcarriers are support matrices that allow cells to be cultured in three dimensions instead of a traditional flat surface, leading to markedly increase in capacity to accommodate more cells in limited volumes. This approach has accelerated production output as the cell growth is largely connected with available surface area for cell adhesion.

Packed bed bioreactors are an attractive method for large scale cell culturing due to the high densities of cells that can be obtained on the large surface area, the flexibility of being able to operate in different batch modes, and the efficiency in executing cell passaging less frequent. In packed bed bioreactors the microcarriers are packed as a bed in the bioreactor while the media continuously circulates within the bioreactor to transfer oxygen and nutrient to cells seeded on the microcarriers. Immobilization of the cells results in high cell densities and a natural means for separating the cells from the culture medium. Products secreted in the culture medium may also be easily recovered.

Since the cells in a packed bed bioreactor system are confined inside the bed comprising the microcarriers, the shear forces (caused by internal flows) that the cells are exposed to are markedly reduced compared to systems where cells are freely exposed in the medium. However, as the microcarriers are constrained in the bed, they are subject to stress from the moving medium. Accordingly, commercially available microcarriers for use with packed bed bioreactors are typically in the form of robust discs that can withstand the shear forces in the bioreactor. The discs are made from non-woven fibers, such as polypropylene, polyester and polyethylene terephthalate (PET), and are thin and relatively dense. The design constraints of the existing microcarriers limits the available surface area and does not present the in-vivo like environment needed by many mammalian cells to proliferate efficiently.

Therefore, scale-up of cell growth in packed bed bioreactors is still non-trivial and may be improved as large quantities of high quality cells are required at a cost-effective mark if any commercialization is to be feasible. At present, there is a common understanding that not only the available surface area, but also the local spatial environment that the cells experience during culture is important for the magnitude and quality of the end cellular product. If the cells are not exposed to sufficient attachment sites and a microenvironment mimicking their natural surroundings, then cell signaling will be hampered and the cells will differentiate poorly.

With biomanufacturing of biologics evolving rapidly there is shaping a picture that scaling of production will be the bottleneck of the biologics' revolution in the coming years. Effective microcarriers which are suitable for use with packed bed bioreactors are at the forefront of solving this problem and assist in accelerating commercialization of biologics.

Thus, there is an unmet need for provision of new improved microcarriers that provides a high quality and scalable option for cell culturing in packed bed bioreactors.

Hence, it would be advantageous to provide a sustainable microcarrier material that offers an increased surface area to the cells in culture, replicates the extracellular environment found in the human body, and which can withstand shear forces generated by flow of medium.

Specifically, it would be advantageous to provide a simple and scalable method for producing an improved microcarrier material that may be utilised all the way from stock culture to bioreactor production.

### SUMMARY OF THE INVENTION

The microcarriers presented herein are based on cellulose nanofibers that in processed form provide a matrix with an increased surface area for cell growth. The cellulose nanofibers offer a scaffold matrix with physical properties that mimic the collagen and elastin fiber structures that make up the human extracellular matrix. Accordingly, cells grown on the microcarrier presented herein adopt physiological characteristics which resemble the native tissue from which they originate, leading to improved intercellular communications, which in turn results in a recovery or maintenance of in vivo functions.

The microcarrier presented herein is based on a mouldable and resilient material that can be integrated into any cell culture systems. It is also durable and can withstand the shear forces that it is subjected to in a packed bed bioreactor system.

Thus, an object of the present invention relates to the provision of a microcarrier suitable for use in a packed bed bioreactor and with the capacity to increase growth of cells.

Another object of the present invention relates to provision of a simple method for producing an improved microcarrier that is scalable for industrial production.

Thus, an aspect of the present invention relates to a method of preparing a nanofibrous cellulose scaffold, said method comprising the steps of:
(i) providing an initial cellulose nanofiber material,
(ii) dividing said initial cellulose nanofiber material into a processed cellulose nanofiber material,
(iii) crosslinking the processed cellulose nanofiber material, and
(iv) drying the processed cellulose nanofiber material,
   thereby providing the nanofibrous cellulose scaffold.

Another aspect of the present invention relates to a nanofibrous cellulose scaffold obtainable from a method as described herein.

Yet another aspect of the present invention relates to a nanofibrous cellulose scaffold comprising a processed cellulose nanofiber material with a mean length of the cellulose nanofibers of less than about 250 μm, and wherein the processed cellulose nanofiber material is crosslinked with a crosslinking agent.

A further aspect of the present invention relates to a microcarrier comprising a nanofibrous cellulose scaffold as described herein.

A still further aspect of the present invention relates to use of a nanofibrous cellulose scaffold as described herein as a microcarrier for cell culturing.

An even further aspect of the present invention relates to a cell culturing device comprising a container loaded with a nanofibrous cellulose scaffold or a microcarrier as described herein.

Another aspect of the present invention relates to a method for cell culturing comprising the steps of:
(i) providing a cell culturing device as described herein,
(ii) adding a composition comprising a cell population to the cell culturing device,
(iii) incubating the cell population to provide a proliferated cell population, and
(iv) optionally, extracting the proliferated cell population from the cell culturing device.

### BRIEF DESCRIPTION OF THE FIGURES

FIG. 1 shows (A) Scanning electron microscopy (SEM) image of the nanofibrous cellulose scaffold. (B) FTIR spectrum of crosslinked cellulose. (C) Comparison of FTIR spectra of (top to bottom) cellulose acetate, regenerated cellulose, QA functionalized and crosslinked cellulose.

FIG. 2 shows (A) Comparison of a traditional, cut, nanofibrous sheet (left) and a moulded nanofibrous cellulose scaffold microcarrier (right). (B) Nanofibrous cellulose scaffold microcarrier moulded to fit lab-scale bioreactors (left) and pilot-scale bioreactors (right). (C-D) Force (N) versus deformation (%) behaviour of the nanofibrous cellulose scaffold (Sponge QA 2%).

FIG. 3 shows examples of fibers cut by laser. (A) The cellulose material cut by laser results in pieces of cellulose that trap air bubbles and float. (B) Electrospun cellulose material cut with a laser. The laser burns the cellulose sheets. (C) SEM images of cellulose nanofibers cut by laser. The cellulose nanofibers are melted and fused together.

FIG. 4 shows microscopy images of cellulose nanofibers divided by blending (A-E) or by dispersing (F-J) for different amounts of times.

FIG. 5 shows the difference between blending and dispersing the cellulose nanofibers. (A) Nanofiber length as a function of means of dividing and the duration of dividing (0.5-10 min). Nanofiber length is determined from SEM images using ImageJ software. (B) Representative SEM image of cellulose nanofibers after 10 minutes of blending. (C) Representative SEM image of cellulose nanofibers after 10 minutes of dispersing.

FIG. 6 shows determination of cellulose nanofiber diameters. (A-B) SEM micrographs of cellulose nanofibers after 1 hour dispersing at 18000 rpm. (C) Histogram depicting the cellulose nanofiber diameter distribution of five separate samples. Approximately 2500 individual cellulose nanofibers were measured using ImageJ software.

FIG. 7 shows testing of viability of cells grown on microcarriers of the nanofibrous cellulose scaffold or cellulose sheets. (A) Experimental layout of a 24-well plate with microcarrier sample allocation indicated. (B) Viability of HEK293T cells cultured on microcarriers. Cell viability is depicted as a percentage compared to a live and dead control. Back bars represent the 24 hours timepoint and grey bars represent the 48 hours timepoint.

FIG. 8 shows representative images of cells grown on each microcarrier. (A) Images of wells representing (left to right) sheet plain, sponge plain, sheet plasma and sponge plasma 0.5%, respectively. (B) Images of wells representing (left to right) sheet QA, sponge QA 0.5%, sponge QA 1.5% and sponge CMC, respectively.

FIG. 9 shows configurations of microcarriers positioned as stacked layers for use in a packed bed bioreactor. (A) Traditional setup with thin microcarrier discs placed between support layers. (B) Nanofibrous cellulose scaffold in form of thick discs placed between support layers. (C) Stacked layers of microcarriers arranged on a shaft.

FIG. 10 shows scanning electron microscopy (SEM) images of electrospun nanofibers; (A) PCL, (B) PLA, (C) PLA/PCL, and (D) cellulose. All images are recorded at 600× magnification, scale bar is 50 μm.

FIG. 11 shows representative photographs (left) and SEM images (right) of different electrospun nanofibers subsequent to 5 minutes of mixing in a blender. (A-B) PCL, (C-D) PLA, (E-F) PLA/PCL. Scale bar of SEM images is 200 μm.

FIG. 12 shows representative photographs (left) and SEM images (right) of different electrospun nanofibers subsequent to 5 minutes of mixing in a disperser. (A-B) PCL, (C-D) PLA, (E-F) PLA/PCL. Scale bar of SEM images is 50 μm.

FIG. 13 shows scanning electron microscopy (SEM) images of cellulose nanofibers blended (left) or dispersed (right). The cellulose nanofibers were mixed for different periods of time; 1 min (A-B), 5 min (C-D), 15 min (E-F) or 60 min (G-H).

FIG. 14 shows histograms of size (length) distributions of cellulose nanofibers that have been divided by (A) blending or (B) dispersing. The histograms are for samples divided for 1, 5, 15, or 60 min (left to right). Nanofiber lengths are displayed as relative frequency of mean nanofiber lengths in bins of 200 μm.

FIG. 15 shows (top) nanofibrous cellulose scaffolds prepared at different cellulose concentrations; (A) 2%, (B) 1%, (C) 0.5%, (D) 0.25%, (E) 0.125%, (F) 0.06%, (G) 0.03%, (H) 0.015%. (middle) Time-course quantification of concentration of HEK293 cells cultured on crosslinked and functionalized nanofibrous cellulose scaffold of different cellulose concentrations. Data is presented as means with each data point being a technical replicate (n=3). (bottom) Sections of nanofibrous cellulose scaffolds displaying the spatial distribution of DAPI-stained cells within the optically cleared porous scaffold at the 72 hours timepoint. Scale bars are 250 μm.

FIG. 16 shows quantification of concentration of HEK293 cells cultured for 72 hours on different functionalized nanofibrous cellulose scaffolds with different crosslinking agent to cellulose ratios; low (3.8*10⁻³ mol %/mol %), medium (19.0*10⁻³ mol %/mol %), high (94.9*10⁻³ mol %/mol %). Data are presented as means with each data point being a technical replicate (n=3).

FIG. 17 shows discs of the nanofibrous cellulose scaffold made with different cellulose concentrations; (I) 0.03%, (II) 0.06%, (III) 0.125%, (IV) 0.25%, and different volumes in the mould; (A) 25 ml, (B) 17 ml, (C) 10 ml, (D) 3 ml.

FIG. 18 shows (A) the setup for mechanical testing; (left) scaffold mounted between two support discs on stirring rod, (middle) stirring rod immersed in 500 ml beaker, (right) visible breakage of sample IIB (0.06% cellulose and 17 ml volume). (B) Recovery of scaffolds after stirring in mockup reactor; samples were as follows according to the reference numbers (A) IA, (B) IIA, (C) IIB, (D) IIIA, (E) IIIB, (F) IIIC, (G) IVA, (H) IVB, (I) IVC.

FIG. 19 shows cell growth curve and glucose consumptions over 5 days of culture in 1 L commercial bioreactor using discs of crosslinked nanofibrous cellulose scaffolds.

### DETAILED DESCRIPTION OF THE INVENTION

### Definitions

Prior to outlining the present invention in more details, a set of terms and conventions is first defined:

### Nanofiber

In the present context, the term “nanofiber” refers to fibers with diameters in the range of 10-2000 nm. The fibers may be generated from different types of polymers, such as cellulose.

### Cellulose Nanofiber Material

In the present context, the term “cellulose nanofiber material” refers to an initial material prepared from cellulose. The cellulose nanofiber material may be prepared by any method suitable for preparing sheets of cellulose that can subsequently be processed as described herein to provide the nanofibrous cellulose scaffold. These methods include, but are not limited to, electrospinning, meltblowing, and drawing of cellulose nanofibers.

Preferably, the cellulose nanofiber material is prepared by electrospinning of cellulose nanofibers. Electrospinning may be performed from a solution of cellulose acetate.

### Container

In the present context, the term “container” refers to any delimited vessel suitable for culturing of cells. The container is preferably a conventional culturing vessel, including, but not limited to, a bioreactor, a cell culturing plate, a cell culture bottle, a spinner flask, a shaker flask, and a roller bottle, a petri dish, and a tube.

The nanofibrous cellulose scaffold as described herein is readily scalable and the container may therefore be of any volume suitable for culturing cells.

### Dispersing

In the present context, the term “dispersing” refers to the process of dividing the cellulose nanofiber material by means of a disperser. Dispersing is preferably performed in a liquid.

In the present context, a disperser is a high-speed mixing device that can comminute a solid, such as a cellulose sheet, into smaller fragments. The disperser comprises one or more heads which constitute the means of dividing the cellulose nanofiber material. The head may be in the form of a disc blade. The head of the disperser may comprise a blade with a propeller design. The disperser creates a turbulent flow and a vortex which ensures homogenous dividing of the cellulose nanofiber material into smaller fragments, i.e. cellulose nanofibers of reduced length compared to the initial cellulose nanofiber material.

Any type of disperser may be used, including, but not limited to, laboratory high-speed disperser, pilot-scale high-speed dispersers, and production-scale high-speed dispersers. The disperser can be chosen according to the batch size to be divided, and the diameter of the disperser blades are adjusted accordingly. The disperser blade may be raised and lowered during dispersing to eliminate stratification, such mechanism may be automatic. For large batches the disperser may be a floor-mounted or tank-mounted model and/or be a multishaft model.

### Crosslinking Agent

In the present context, the term “crosslinking agent” refers to any chemical or biological molecule capable of connecting multiple cellulose nanofibers to each other. Preferably, the crosslinking agent can connect cellulose nanofibers via reaction with hydroxyl groups of the cellulose backbone to create covalently crosslinked cellulose nanofibers.

Thus, a crosslinking agent may be a molecule comprising at least two functional groups capable of reacting with hydroxyl groups.

### Functional Moiety

In the present context, the term “functional moiety” refers to a chemical or biological group or molecule positioned on the nanofibrous cellulose scaffold, and which interacts with the cells associated with scaffold. A functional moiety may interact with the cells via interactions including, but not limited to, electrostatic interactions, affinity, and hydrophobicity/hydrophilicity. The functional moiety may promote attachment/adhesion of cells to the nanofibrous cellulose scaffold, induce cell differentiation and proliferation, and/or assist maintenance of in vivo cellular functions.

Chemical moieties may have one or more positive or negative charges to induce electrostatic interaction with the charged cell membranes. Examples hereof include, but are not limited to, quaternary ammonium (QA), carboxymethyl (CM), and diethylaminoethyl (DEAE). For many cells, the cell membrane would be negatively charged, and electrostatic interactions would be induced for nanofibrous cellulose scaffolds functionalized with positively charged functional moieties, such as QA or DEAE.

Biological moieties may be any type of biological molecule that can secure cell attachment to the nanofibrous cellulose scaffold, including but not limited to, lipid anchors, cell-adhesion molecules (CAMs), antigens, receptors, and antibodies. CAMs include integrins, immunoglobulins, cadherins, and selectins. Biological moieties may also assist cellular differentiation through receptor-ligand interactions, antigen-antibody interactions, protein interactions, and/or immune stimulatory signalling.

### Adherent Cell

In the present context, the term “adherent cells” refers to any cell that requires a surface or artificial substrate, such as a microcarrier, to form an adherent cell culture. Preferably, the adherent cell is derived from a solid tissue.

Adherent culture is to be distinguished from suspension culture in which cells are grown freely floating in suspension.

### Mean Diameter (of Cellulose Nanofiber)

In the present context, the term “mean diameter” refers to the average diameter of cellulose nanofibers in the nanofibrous cellulose scaffold. The mean diameter may be determined from SEM images of the nanofibrous cellulose scaffold. Preferably, the mean diameter is determined from measurement of at least 100 individual nanofibers within the sample, e.g. by use of image analysis software, such as ImageJ.

The mean diameter of the cellulose nanofibers can be adjusted in the process of preparing the initial cellulose nanofiber material. This may be achieved by varying the parameters of e.g. the electrospinning process.

Preferably, the mean diameter of the cellulose nanofibers in the nanofibrous cellulose scaffold is from about 250 nm to about 750 nm, such as about 400 nm to about 600 nm.

### Mean Length (of Cellulose Nanofiber)

In the present context, the term “mean length” refers to the average length of cellulose nanofibers in the nanofibrous cellulose scaffold. The mean length may be determined as the volume weighted mean value (D[4,3]) measured by light scattering, e.g. on a Malvern Mastersizer S. D[4,3] is also known as the De Brouckere mean value.

The mean fiber length in a sample may be determined using the following settings on a Malvern Mastersizer S:
Range lens: 300RF mm
Presentation: 3OHD
Analysis model: Polydisperse
Particle refractive index: (1.5295, 0.1000)
Dispersant refractive index: (1.33000)
Density: 1.5000 g/cm³

Preferably, the mean length of the cellulose nanofibers is from about 30 μm to about 250 μm, such as about 40 μm to about 200 μm, such as about 50 μm to about 150 μm, preferably about 60 μm to about 100 μm.

### Surface Area

In the present context, the term “surface area” refers to the Brunauer-Emmett-Teller (BET) surface area. The BET surface area may be determined by measurement of the physiosorption of a gas, usually nitrogen, to give a value of the sample. The BET method can accurately determine the surface area of the nanofibrous cellulose scaffold since the gas molecules can travel within the nanofibrous matrix to probe also interior surfaces.

The surface area is given as area per unit mass (e.g. cm²/g) and can be measured according to ISO 9277:2022—Determination of the specific surface area of solids by gas adsorption—BET method.

### Degree of Substitution (DS)

In the present context, the term “degree of substitution (DS)” refers to the average number of functional moieties attached per base unit of the condensation polymer cellulose. The base unit of cellulose is β(1→4) linked D-glucose, which comprise three hydroxyl groups that may be subjected to substitution. Accordingly, the theoretical maximum value of DS is 3.

Degree of substitution (DS) may be determined using the following formula:

DS=(162N/(1400−cA×N))

where 162 is the molecular weight of the anhydrous glucose unit (AGU); N is the percentage of nitrogen; CA is the molecular weight of the cationic reagent.

Substitution of the cellulose nanofibers can also be quantified as equivalent of charge per base unit mass of cellulose, and is given in units of meq/g. This value can be determined by zeta potential measurements, pH titration or electrokinetic chromatography.

### Dead Volume

In the present context, the term “dead volume” refers to the volume occupied by the microcarrier when culturing cells. Ideally, the dead volume is minimised to allow for more cell proliferation per volume in the container used for culturing cells.

### Mercerization

In the present context, the term “mercerization” refers to a process comprising swelling of the cellulose nanofiber material in an aqueous or ethanolic NaOH solution to break internal hydrogen bonds of cellulose and increase the number of available hydroxyl groups (—OH).

### Microcarrier

In the present context, the term “microcarrier” refers to any support matrix upon which adherent cells may grow in adherent culture.

### Packed Bed Bioreactor

In the present context, the term “packed bed bioreactor” refers to a bioreactor wherein the microcarrier is immobilised in a bed within the bioreactor. Fresh medium is continuously circulating within the system. The means for providing circulation (or agitation) within the bioreactor include, but is not limited to, a stirrer, such as an impeller, or a pump. Cells are seeded on the microcarrier in the bed.

Packed bed bioreactors fall in two overall categories; fixed bed bioreactors where the packed bed is stationary, and dynamic bed bioreactors wherein the packed bed is in motion.

### Dry Continuous Material

In the present context, the term “dry continuous material” refers to a dry material that can be produced as a single entity that extends significantly in three dimensions. Extending significantly includes, but is not limited to, extending at least about 0.1 mm in all three dimensions.

A dry continuous material is to be distinguished from materials that only gain their volumetric form from adding individual entities together. Examples of non-continuous materials include, but are not limited to, a high density population of solid spherical particles, or a collection of stacked discs.

### Resilient

In the present context, the term “resilient” refers to the ability of a material to return to its original shape and size after being subjected to external forces or stresses. Thus, a resilient material is able to absorb energy when it is deformed, and then release that energy when the deforming force is removed, causing the material to return to its original shape. This property is often referred to as “elasticity” or “elastic deformation”. A resilient material is both compressible and has a sufficient elasticity allowing the material to decompress and expand back to the initial shape when the force on the material is removed.

Accordingly, a resilient material can be forced into a container or void of a smaller volume, and adopt the shape of that container or void. This change of shape is, in principal, reversible in nature, as release from the smaller volume or void will return the material to its original shape and size.

### Compressive Strength

In the present context, the term “compressive strength” refers to the ability of a material to withstand a compressive load. The compressive strength may be given by the force required to cause a specific deformation. The value can be given in units of mm/N.

As a benchmark, the compressive strength of a material can be presented as the force necessary to obtain 60% deformation of the material.

The compressive strength can be measured according to ISO 604:2002—Plastics—Determination of compressive properties.

### About

Wherever the term “about” is employed herein in the context of amounts, for example absolute amounts, such as numbers, purities, weights, sizes, etc., or relative amounts (e.g. percentages, equivalents or ratios), timeframes, and parameters such as temperatures, pressure, etc., it will be appreciated that such variables are approximate and as such may vary by +10%, for example #5% and preferably #2% (e.g. #1%) from the actual numbers specified. This is the case even if such numbers are presented as percentages in the first place (for example ‘about 10%’ may mean±10% about the number 10, which is anything between 9% and 11%).

### Nanofibrous Cellulose Scaffold

Herein are described a nanofibrous cellulose scaffold that may be used as a microcarrier for culturing cells. The nanofibrous cellulose scaffold has properties, such as a large surface area and low dead volume, which unlocks a significant increase in cell proliferation per unit culturing volume. Moreover, the mechanical properties of the scaffold makes it particularly advantageous for use in packed bed bioreactors to withstand forces from internal flows. Importantly, the nanofibrous cellulose scaffold can be produced by a simple and cost-effective method yielding a commercially attractive and readily scalable end-product.

Thus, an aspect of the present invention relates to a method of preparing a nanofibrous cellulose scaffold, said method comprising the steps of:
(i) providing an initial cellulose nanofiber material,
(ii) dividing said initial cellulose nanofiber material into a processed cellulose nanofiber material,
(iii) crosslinking the processed cellulose nanofiber material, and
(iv) drying the processed cellulose nanofiber material,
   thereby providing the nanofibrous cellulose scaffold.

The resulting nanofibrous cellulose scaffold mimics the extracellular matrix (ECM) and has a high surface area due to the homogeneous distribution of cellulose nanofibers. The ECM is important for survival, proliferation, differentiation and migration of the cells, and microcarriers mimicking the properties of the ECM is therefore considered a step in the direction of in vivo-like cell culturing.

The flexible nature of the cellulose nanofibers allows them to form a network of strands that efficiently exploit the space occupied and reduces the microcarrier dead volume that is not accessible to cells. Accordingly, more surface area can be packed into a smaller volume which is advantageous for use in cell culturing containers wherein only a finite volume is available.

The compression profile of the nanofibrous cellulose scaffold is advantageous in that the flexible material can easily be adapted for any cell culturing platform, while the mechanical strength of the material ensures that it is not damaged or dissolved by the internal media flow in packed bed bioreactors.

The present method therefore provides an improved microcarrier material that may be used for increasing cell growth and product yield.

Crosslinking of the individual cellulose nanofibers is preferably achieved by the addition of a crosslinking agent. The crosslinking agent is not limited to any specific type of crosslinking agent. The cellulose backbone comprise hydroxyl groups that may be used to bridge the nanofibers via the crosslinking agent. Thus, the crosslinking agent preferable has functional groups that may react with the hydroxyl groups to create covalent bonds.

Therefore, an embodiment of the present invention relates to the method as described herein, wherein the processed cellulose nanofiber material is crosslinked by addition of a crosslinking agent.

Another embodiment of the present invention relates to the method as described herein, wherein the crosslinking agent comprises at least two functional groups that can react with hydroxyl groups to form covalent bonds.

A further embodiment of the present invention relates to the method as described herein, wherein the crosslinking agent comprises at least two functional groups selected from the group consisting of carboxylic acids, aldehydes, epoxides, halides, anhydrides, silanes, and azetidinium.

Yet another embodiment relates to the method as described herein, wherein the crosslinking agent comprises at least two azetidinium groups.

Some crosslinking agents have been identified as particular useful for crosslinking of cellulose nanofibers. In particular, it is demonstrated herein that crosslinking with polyamide epichlorohydrin results in nanofibrous cellulose scaffolds with large surface areas and great mechanical properties.

Therefore, an embodiment of the present invention relates to the method as described herein, wherein the processed cellulose nanofiber material is crosslinked by addition of a crosslinking agent selected from the group consisting of epichlorohydrin, formaldehyde dimethyl acetate (FDA), maleic anhydride (MAL), 3-glycidoxypropyltrimethoxysilane (GPS), citric acid, and 1,2,3,4-butanetetracarboxylic acid.

A preferred embodiment of the present invention relates to the method as described herein, wherein the crosslinking agent is a polyamide epichlorohydrin resin.

Another preferred embodiment of the present invention relates to the method as described herein, wherein the crosslinking agent is a hexanedioic acid, polymer with N1-(2-aminoethyl)-1,2-ethanediamine and 2-(chloromethyl) oxirane.

Hexanedioic acid is also known as adipic acid and has the chemical formula (CH₂)₄(COOH)₂. Epichlorohydrin is also known as 2-(chloromethyl) oxirane and is an organochlorine compound and an epoxide. N1-(2-aminoethyl)-1,2-ethanediamine is also known as diethylenetriamine and is an organic compound with the formula HN(CH₂CH₂NH₂)₂. It is N-alkylated upon reaction with epoxide groups forming crosslinks.

Accordingly, a preferred embodiment of the present invention relates to the method as described herein, wherein the crosslinking agent is adipic acid diethylenetriamine epichlorohydrin copolymer.

It is to be understood that copolymers may also be referred to as resins.

The amount of crosslinking agent may be varied to modify the properties of the nanofibrous cellulose scaffold. For example, increasing the amount of crosslinking agent will create more dense and mechanically strong material. It has been found that certain ratios of cellulose nanofiber material to crosslinking agent promotes properties such as high available surface area and sufficient mechanical strength.

Thus, an embodiment of the present invention relates to the method as described herein, wherein the ratio of crosslinking agent to processed cellulose nanofiber material is in the range of about 1:34 (vol %/wt %) to about 1:1 (vol %/wt %), such as about 1:30 (vol %/wt %) to about 1:5 (vol %/wt %), such as about 1:25 (vol %/wt %) to about 1:10 (vol %/wt %), preferably about 1:20 (vol %/wt %) to about 1:15 (vol %/wt %), more preferably about 1:17 (vol %/wt %).

Another embodiment of the present invention relates to the method as described herein, wherein the ratio of crosslinking agent to processed cellulose nanofiber material is in the range of about 1:136 (vol %/wt %) to about 1:2 (vol %/wt %), such as about 1:68 (vol %/wt %) to about 1:5 (vol %/wt %), such as about 1:34 (vol %/wt %) to about 1:10 (vol %/wt %), preferably about 1:20 (vol %/wt %) to about 1:15 (vol %/wt %), more preferably about 1:17 (vol %/wt %).

A further embodiment of the present invention relates to the method as described herein, wherein the ratio of crosslinking agent to processed cellulose nanofiber material is in the range of about 1:50 (vol %/wt %) to about 4:1 (vol %/wt %), such as about 1:20 (vol %/wt %) to about 2:1 (vol %/wt %), such as about 1:10 (vol %/wt %) to about 1:1 (vol %/wt %), such as about 1:5 (vol %/wt %) to about 1:2 (vol %/wt %).

A preferred embodiment of the present invention relates to the method as described herein, wherein the ratio of crosslinking agent to processed cellulose nanofiber material is about 1:5 (vol %/wt %).

Another preferred embodiment of the present invention relates to the method as described herein, wherein the ratio of crosslinking agent to processed cellulose nanofiber material is about 1:2.5 (vol %/wt %).

A further embodiment of the present invention relates to the method as described herein, wherein the ratio of crosslinking agent to processed cellulose nanofiber material is less than about 1:1 (vol %/wt %), such as less than about 1:15 (vol %/wt %), less than about 1:2 (vol %/wt %).

The amount of crosslinking agent may also be presented as the mol % of crosslinking agent per mol % processed cellulose nanofiber material. The ratio is relevant because it sets out how many crosslinking units that connects with glucose units within the nanofibrous cellulose scaffold. By altering the number of crosslinkers per glucose unit, the properties of the scaffold will change. Thus, a higher mol %/mol % ratio may increase mechanical strength and reduce porosity. Herein, the mol % for the processed cellulose nanofiber material is given per glucose unit.

Therefore, an embodiment of the present invention relates to the method as described herein, wherein the processed cellulose nanofiber material is crosslinked with a crosslinking agent in a crosslinking agent to processed cellulose nanofiber material ratio in the range of about 2*10⁻³ mol %/mol % to about 175*10⁻³ mol %/mol %, such as about 5*10⁻³ mol %/mol % to about 100*10⁻³ mol %/mol %, such as about 10*10⁻³ mol %/mol % to about 80*10⁻³ mol %/mol %, such as about 15*10⁻³ mol %/mol % to about 50*10⁻³ mol %/mol %, such as about 25*10⁻³ mol %/mol % to about 40*10⁻³ mol %/mol %.

Another embodiment of the present invention relates to the method as described herein, wherein the processed cellulose nanofiber material is crosslinked with a crosslinking agent in a crosslinking agent to processed cellulose nanofiber material ratio in the range of about 15*10⁻³ mol %/mol % to about 80*10⁻³ mol %/mol %, such as about 25*10⁻³ mol %/mol % to about 70*10⁻³ mol %/mol %, such as about 30*10⁻³ mol %/mol % to about 50*10⁻³ mol %/mol %, such as about 35*10⁻³ mol %/mol % to about 40*10⁻³ mol %/mol %.

It has been found that for some varieties of the nanofibrous cellulose scaffold it can be advantageous to decrease the crosslinking agent to processed cellulose nanofiber material ratio. Without being bound by theory, it is contemplated herein that the lower crosslinking degree may assist the migration of cells within the scaffold, resulting in better cell growth.

Thus, an embodiment of the present invention relates to the method as described herein, wherein the processed cellulose nanofiber material is crosslinked with a crosslinking agent in a crosslinking agent to processed cellulose nanofiber material ratio of less than about 90*10⁻³ mol %/mol %, such as less than about 75*10⁻³ mol %/mol %, such as less than about 50*10⁻³ mol %/mol %, such as less than about 40*10⁻³ mol %/mol %, such as less than about 30*10⁻³ mol %/mol %, such as less than about 25*10⁻³ mol %/mol %, such as less than about 20*10⁻³ mol %/mol %.

A preferred embodiment of the present invention relates to the method as described herein, wherein the processed cellulose nanofiber material is crosslinked with a crosslinking agent in a crosslinking agent to processed cellulose nanofiber material ratio in the range of about 30*10⁻³ mol %/mol % to about 50*10⁻³ mol %/mol %.

It is to be understood that the mol % of the cellulose material refers to the mol % per glucose unit in the processed cellulose nanofiber material.

A further embodiment of the present invention relates to the method as described herein, wherein the crosslinking step is performed at a temperature in the range of about 15° C. to about 25° C., preferably at about 20° C.

Dividing of the initial cellulose nanofiber material is important as it ensures a homogeneous distribution of the cellulose nanofibers in the final scaffold. The shorter cellulose nanofibers also significantly reduces the risk of entanglement of the nanofibers and formation of clusters of nanofibers, which leads to less available surface area exposed to the cells and increased risk of blocking the impeller of a bioreactor. Moreover, larger entanglements or clusters of cellulose nanofibers also render the final nanofibrous cellulose scaffold difficult to handle and may cause clogging of tubing or during pipetting. As part of the upstream biomanufacturing process, pipetting or tapping from the bioreactor is continuously performed in order to perform cell counting, viability and yield results. Therefore, a microcarrier solution that has even a small risk of clogging will not be viable.

It has been found that dispersing the initial cellulose nanofiber material results in a homogeneous material that is suitable for use as a microcarrier. Without being bound by theory, it is contemplated that dispersing the initial cellulose nanofiber material results in a distribution which are spaced so as to allow multiple attachment points for the cells leading to improved proliferation.

Accordingly, an embodiment of the present invention relates to the method as described herein, wherein dividing said initial cellulose nanofiber material is achieved by dispersing.

Another embodiment of the present invention relates to the method as described herein, wherein dispersing is performed with a high-speed disperser.

A further embodiment of the present invention relates to the method as described herein, wherein dividing said initial nanofiber material comprises a step of cutting the initial nanofiber material with a disperser.

Increasing the dispersing time reduces the mean length of the cellulose nanofibers in the nanofibrous cellulose scaffold. In particular, it is advantageous to disperse the material for at least a couple of minutes to reduce nanofiber entanglement and cluster formation. Also, without being bound by theory, it is contemplated that longer cellulose nanofibers are not as easily substituted on the hydroxyl groups, thereby leading to a lower degree of substitution (DS) of functional moieties.

Thus, an embodiment of the present invention relates to the method as described herein, wherein dispersing is performed for at least 2 min, such as at least 5 min, such as at least 10 min.

Another embodiment of the present invention relates to the method as described herein, wherein dispersing is performed for at least 2 min, such as at least 5 min, such as at least 10 min, such as at least 15 min, such as at least 20 min, such as at least 30 min, such as at least 40 min, such as at least 50 min, such as at least 60 min, such as at least 90 min, such as at least 120 min.

A further embodiment of the present invention relates to the method as described herein, wherein dispersing is performed for a period of time in the range of 2 min to 120 min, such as 2 min to 90 min, such as 5 min to 60 min, such as 10 min to 60 min, such as 15 min to 60 min.

A preferred embodiment of the present invention relates to the method as described herein, wherein dispersing is performed for at least 5 min.

Another preferred embodiment of the present invention relates to the method as described herein, wherein dispersing is performed for at least 15 min.

A still further preferred embodiment of the present invention relates to the method as described herein, wherein dispersing is performed for at least 60 min.

Dispersing of the initial cellulose nanofiber material for at least 15 min, and even at least 60 min, can be advantageous as it reduces entanglement and cluster formation of the cellulose nanofibers.

Preferably dispersing is performed at high speed, such as at about 18000 rpm. The speed may be adjusted depending on the type of disperser. Any type of disperser may be used, including, but not limited to, laboratory high-speed disperser, pilot-scale high-speed dispersers, and production-scale high-speed dispersers. The speed may be adjusted to produce a good vortex in the solution, and can depend on volume and viscosity of the solution.

Therefore, an embodiment of the present invention relates to the method as described herein, wherein dispersing is performed at a speed in the range of about 10000 rpm to about 30000 rpm, such as at about 12000 rpm to about 25000 rpm, such as about 15000 rpm to about 20000 rpm.

Yet another embodiment of the present invention relates to the method as described herein, wherein dispersing is performed at at least 15000 rpm, such as at at least 20000 rpm, such as at least 25000 rpm.

During the dividing step, the cellulose nanofibers are reduced in length. It is important that the cellulose nanofibers are not too long as it will cause entanglement of the nanofibers and cluster formation. Cells are not able to penetrate and migrate into these tight clusters of entangled fibers and thereby a portion of the large surface area of the nanofibers are lost.

Therefore, an embodiment of the present invention relates to the method as described herein, wherein the mean length of the cellulose nanofibers in said processed cellulose nanofiber material is shorter than the mean length of the cellulose nanofibers in said initial cellulose nanofiber material.

Another embodiment of the present invention relates to the method as described herein, wherein the mean length of the cellulose nanofibers in said processed cellulose nanofiber material is less than 250 μm, such as less than about 200 μm, such as less than about 150 μm, such as less than about 120 μm, such as less than about 100 μm, such as less than about 80 μm.

A further embodiment of the present invention relates to the method as described herein, wherein the mean length of the cellulose nanofibers in said processed cellulose nanofiber material is in the range of about 30 μm to about 250 μm, such as about 40 μm to about 200 μm, such as about 50 μm to about 150 μm, preferably about 60 μm to about 100 μm.

A still further embodiment of the present invention relates to the method as described herein, wherein the mean length of the cellulose nanofibers in said processed cellulose nanofiber material is in the range of about 70 μm to about 120 μm.

The dividing step may be performed under cooling to lower the ductility of the cellulose nanofibers, making them more brittle and easier to divide. The cooling may occur before or during the dividing of the initial cellulose nanofiber material. Cooling may comprise cooling of the container in which the initial cellulose nanofiber material is held during the dividing step or cooling the initial cellulose nanofiber material by exposure to a coolant, such as liquid hydrogen, liquid helium and/or liquid nitrogen, or by keeping the initial cellulose nanofiber material in a fridge or freezer immediately before the dividing step.

Thus, an embodiment of the present invention relates to the method as described herein, wherein the initial cellulose nanofiber material is cooled before or during the dividing step.

Cellulose sheets, if prepared by electrospinning, may be highly static and difficult to handle. Therefore, the initial cellulose nanofiber material may conveniently be provided as a liquid sample that is ready for processing, e.g. by dispersing.

Thus, an embodiment of the present invention relates to the method as described herein, wherein the initial cellulose nanofiber material is provided as a liquid sample.

Another embodiment of the present invention relates to the method as described herein, wherein the solvent of the liquid sample comprises water and/or ethanol.

A further embodiment of the present invention relates to the method as described herein, wherein the solvent of the liquid sample comprises ethanol.

The properties of the nanofibrous cellulose scaffold (e.g. after drying and moulding) may be guided by the density of cellulose nanofibers that the network comprises subsequent to removal of liquid. For example, addition of lower concentrations of cellulose nanofibers will provide a more porous network wherein the cells may easier migrate within. In contrast, higher concentrations of cellulose nanofibers result in greater mechanical strength of the material. These properties are also affected by the degree of crosslinking. Some advantageous combinations of cellulose content and crosslinking degree that provides sufficient mechanical strength of the material to survive shear forces in a packed bed bioreactor and at same time provide high available surface area has been identified herein.

Thus, an embodiment of the present invention relates to the method as described herein, wherein the concentration of cellulose nanofibers in the processed cellulose nanofiber material is about 0.1 wt % to about 10 wt %, such as about 0.15 wt % to about 8 wt %, such as about 0.2 wt % to about 5 wt %, such as about 0.2 wt % to about 3 wt %, such as about 0.25 wt % to about 2 wt %, preferably about 0.3 wt % to about 1 wt %, with respect to the total weight of the liquid sample.

Another embodiment of the present invention relates to the method as described herein, wherein the concentration of cellulose nanofibers in the processed cellulose nanofiber material is about 0.1 wt % to about 5 wt %, such as about 0.15 wt % to about 2.5 wt %, such as about 0.25 wt % to about 1.5 wt %, preferably about 1 wt %, with respect to the total weight of the liquid sample.

For some variants of the nanofibrous cellulose scaffold it has been found to be advantageous to retain the cellulose concentration at a low level. Maintaining a low cellulose concentration secures a high porosity of the resulting nanofibrous cellulose scaffold, thereby allowing cells to migrate into the scaffold and utilise the large exposed surface area of the nanofibers.

Therefore, an embodiment of the present invention relates to the method as described herein, wherein the concentration of cellulose nanofibers in the processed cellulose nanofiber material is about 0.01 wt % to about 1 wt %, such as about 0.02 wt % to about 0.5 wt %, such as about 0.03 wt % to about 0.2 wt %.

A preferred embodiment of the present invention relates to the method as described herein, wherein the concentration of cellulose nanofibers in the processed cellulose nanofiber material is about 0.05 wt % to about 0.2 wt %, such as about 0.08 wt % to about 0.17 wt %, such as about 0.1 wt % to about 0.15 wt %.

Another preferred embodiment of the present invention relates to the method as described herein, wherein the concentration of cellulose nanofibers in the processed cellulose nanofiber material is about 0.125 wt %.

Yet another embodiment of the present invention relates to the method as described herein, wherein the concentration of cellulose nanofibers in the processed cellulose nanofiber material is about 0.25 wt % to about 2 wt %, with respect to the total weight of the liquid sample, and the ratio of crosslinking agent to processed cellulose nanofiber material is in the range of about 1:20 (vol %/wt %) to about 1:15 (vol %/wt %).

A further embodiment of the present invention relates to the method as described herein, wherein the concentration of cellulose nanofibers in the processed cellulose nanofiber material is about 0.3 wt % to about 1 wt %, such as about 0.3 wt % to about 0.7 wt %, with respect to the total weight of the liquid sample, and the ratio of crosslinking agent to processed cellulose nanofiber material is in the range of about 1:20 (vol %/wt %) to about 1:15 (vol %/wt %), preferably about 1:17 (vol %/wt %).

A preferred embodiment of the present invention relates to the method as described herein, wherein the concentration of cellulose nanofibers in the processed cellulose nanofiber material is about 0.05 wt % to about 0.2 wt %, such as about 0.1 wt % to about 0.15 wt %, with respect to the total weight of the liquid sample, and the ratio of crosslinking agent to processed cellulose nanofiber material is in the range of about 30*10⁻³ mol %/mol % to about 50*10⁻³ mol %/mol %.

Another preferred embodiment of the present invention relates to the method as described herein, wherein the concentration of cellulose nanofibers in the processed cellulose nanofiber material is about 0.05 wt % to about 0.2 wt %, such as about 0.1 wt % to about 0.15 wt %, with respect to the total weight of the liquid sample, and the ratio of crosslinking agent to processed cellulose nanofiber material is in the range of about 30*10⁻³ mol %/mol % to about 50*10⁻³ mol %/mol %, and the nanofibrous cellulose scaffold is a disc with a thickness in the range of about 5 mm to about 9 mm.

A further preferred embodiment of the present invention relates to the method as described herein, wherein the concentration of cellulose nanofibers in the processed cellulose nanofiber material is about 0.125 wt %, with respect to the total weight of the liquid sample, and the ratio of crosslinking agent to processed cellulose nanofiber material is in the range of about 30*10⁻³ mol %/mol % to about 50*10⁻³ mol %/mol %, and the nanofibrous cellulose scaffold is a disc with a thickness of about 9 mm.

The mean diameter of the cellulose nanofibers can be guided during the preparation of the initial cellulose nanofiber material. This may be achieved by varying the parameters of e.g. the electrospinning or meltblowing process, such as the voltage or heat applied, the speed and type of injection, and/or the rotational speed of the collector drum. The mean diameter of the cellulose nanofibers may be varied depending on the application, e.g. the type of cells to be cultured. It has been found that for many applications, a mean diameter of about 400 nm to about 600 nm, such as about 500 nm, is advantageous.

Accordingly, an embodiment of the present invention relates to the method as described herein, wherein the initial cellulose nanofiber material is prepared by a process selected from the group consisting of electrospinning, meltblowing, drawing, self-assembly, template synthesis, and thermal-induced phase separation.

Another embodiment of the present invention relates to the method as described herein, wherein the initial cellulose nanofiber material is prepared by electrospinning.

A further embodiment of the present invention relates to the method as described herein, wherein the mean diameter of the cellulose nanofibers in said initial cellulose nanofiber material and/or processed cellulose nanofiber material is in the range of about 10 nm to about 2000 nm, such as about 50 nm to about 1500 nm, such as about 100 nm to about 1000 nm, preferably about 250 nm to about 750 nm.

In a preferred embodiment of the present invention relates to the to the method as described herein, wherein the mean diameter of the cellulose nanofibers in said initial cellulose nanofiber material and/or processed cellulose nanofiber material is in the range of about 400 nm to about 600 nm, preferably about 500 nm.

The cellulose nanofibers may be combined with other types of nanofibers to enhance the nanofibrous cellulose scaffold with new properties. This may be beneficial for specific cell types wherein the addition of other types of nanofibers widens the options for mimicking the local extracellular matrix of that particular tissue wherefrom the cells are. The additional polymers may be of natural or synthetic origin. For natural nanofibers, especially types of nanofibers traditionally present in the extracellular environment, such as collagen, may work in synergy with cellulose to mimic the ECM. Additional polymers may also be introduced to modify the mechanical properties of the material.

Therefore, an embodiment of the present invention relates to the method as described herein, wherein the initial cellulose nanofiber material further comprises one or more nanofibers selected from natural polymers or synthetic polymers.

Another embodiment of the present invention relates to the method as described herein, wherein the natural polymers are selected from the group consisting of collagen, silk fibroin, keratin, gelatin and polysaccharides, and combinations thereof.

A further embodiment of the present invention relates to the method as described herein, wherein the synthetic polymers are selected from the group consisting of polycaprolactone (PCL), poly(lactic acid) (PLA), polystyrene, polyurethane (PU), poly(lactic-co-glycolic acid) (PLGA), poly(3-hydroxybutyrate-co-3-hydroxyvalerate) (PHBV), and poly(ethylene-co-vinylacetate) (PEVA), and combinations thereof.

The initial cellulose nanofiber material may be prepared from a cellulose acetate solution, e.g. by electrospinning of a cellulose acetate solution. The resulting cellulose acetate sheets are preferably regenerated to cellulose sheets in a sodium hydroxide bath before any further processing. This treatment opens up hydroxyl groups that may subsequently be used for binding of functional moieties.

Thus, an embodiment of the present invention relates to the method as described herein, wherein the initial cellulose nanofiber material is prepared by electrospinning of a cellulose acetate solution to cellulose acetate sheets.

Another embodiment of the present invention relates to the method as described herein, wherein the cellulose acetate sheets are regenerated to cellulose sheets by treatment with NaOH.

A still further embodiment of the present invention relates to the method as described herein, wherein said initial cellulose nanofiber material is regenerated in a regeneration solution comprising sodium hydroxide.

Another embodiment of the present invention relates to the method as described herein, wherein the regeneration solution comprises sodium hydroxide in the range of about 0.1M to about 1 M, such as about 0.2 M to about 0.8 M, such as about 0.3 M to about 0.7 M, such as about 0.4 M to about 0.6 M, preferably about 0.5 M.

Regeneration of cellulose sheets is preferably performed in an ethanol solution comprising sodium hydroxide. It is possible to use varying amounts of ethanol, such as from 5% vol/vol to 99% vol/vol.

Thus, an embodiment of the present invention relates to the method as described herein, wherein the regeneration solution is an ethanol solution comprising about 5% vol/vol to about 99% vol/vol ethanol, such as about 10% vol/vol to about 95% vol/vol ethanol, such as about 20% vol/vol to about 90% vol/vol ethanol, such as about 30% vol/vol to about 80% vol/vol ethanol, such as about 40% vol/vol to about 70% vol/vol ethanol.

The content of ethanol in the regeneration solution may influence the mechanical properties of the resulting nanofibrous cellulose scaffold. Mechanical properties that can be impacted by the content of ethanol includes elasticity and brittleness. Without being bound by theory, it is contemplated that a more elastic and less stiff material is beneficial for interaction with the cells and to promote proliferation.

Thus, an embodiment of the present invention relates to the method as described herein, wherein the regeneration solution is an ethanol solution comprising at least about 50% vol/vol ethanol, such as at least 60% vol/vol ethanol, such as at least 70% vol/vol ethanol, such as at least 80% vol/vol ethanol, such as at least 90% vol/vol ethanol, preferably about 90% vol/vol ethanol.

The cellulose nanofibers may be functionalized with functional moieties to engraft the nanofibrous scaffold with new properties. The functional moieties substituted onto the nanofibrous cellulose scaffold may include ECM proteins, peptides, and/or charged groups to e.g. increase attachment levels of the cells to the scaffold, promote differentiation of the cells, or assist in the release and isolation of the cells from the scaffold. Functionalization of the cellulose nanofibers may be carried out before or after crosslinking of the cellulose nanofibers. Preferably, the functionalization step is performed before crosslinking.

Thus, an embodiment of the present invention relates to the method as described herein further comprising a step of functionalizing the processed cellulose nanofiber strands by addition of a reagent comprising a functional moiety, said further step immediately preceding or immediately following the crosslinking step (iii).

Another embodiment of the present invention relates to the method as described herein further comprising a step of functionalizing the processed cellulose nanofiber strands by addition of a reagent comprising a functional moiety, said further step preceding the crosslinking step (iii).

Subsequent to dividing of the initial cellulose nanofiber material, the resulting processed material is preferably handled to make it ready for functionalization. Part of the treatment can include filtration, washing and re-suspension of the processed cellulose nanofiber material. Filtration ensures that excess water is removed from the processed cellulose nanofiber material. Washing assists in removal of any acetate ions still present after the previous treatment. Careful washing can assist with improving the texture of the cellulose. When re-suspending the processed cellulose nanofiber material, the concentration of cellulose nanofibers may be adjusted if desired.

Therefore, an embodiment of the present invention relates to the method as described herein, wherein the dividing step (ii) is followed by a step comprising filtration, washing and suspending the processed cellulose nanofiber material.

Another embodiment of the present invention relates to the method as described herein, wherein said filtration comprises sieving of the processed cellulose nanofiber material.

Yet another embodiment of the present invention relates to the method as described herein, wherein the suspension comprises water and/or ethanol.

Prior to functionalization, the cellulose nanofibers are preferably mercerized to improve the substitution of functional moieties onto the cellulose nanofibers. Mercerization is a process in which the cellulose nanofibers are swollen by immersion in a concentrated aqueous NaOH solution, washed with water, and dried. During the mercerization process, the crystal structure of the cellulose nanofiber is transformed from cellulose I to cellulose II. Under the action of concentrated alkaline solutions chemical, physicochemical and structural modifications of cellulose occur. Upon washing and neutralisation cellulose II is formed. As a result of the penetration of the base into the lattice, internal hydrogen bonds are broken and the number of available hydroxyl groups (—OH) in the cellulose nanofiber is increased. It is therefore contemplated that mercerization improves the degree of substitution (DS). The mercerization step may be performed before or after the dividing step.

Therefore, an embodiment of the present invention relates to the method as described herein further comprising a step of mercerization of said processed cellulose nanofiber material.

Another embodiment of the present invention relates to the method as described herein, wherein the mercerization step is immediately before or after the dividing step (ii).

Yet another embodiment of the present invention relates to the method as described herein, wherein said mercerization step comprises addition of NaOH.

A further embodiment of the present invention relates to the method as described herein, wherein concentration of NaOH is in the range of about 0.05 M to about 2 M, such as about 0.1 M to about 1.5 M, such as about 0.25 M to about 0.75 M, preferably about 0.4 M to about 0.6 M.

A still further embodiment of the present invention relates to the method as described herein, wherein the mercerization step is performed for a period of about 1 hour to about 3 hours, preferably about 2 hours.

An even further embodiment of the present invention relates to the method as described herein, wherein the degree of substitution of the functional moiety (DS) is in the range of about 0.01 to about 2.

Another embodiment of the present invention relates to the method as described herein, wherein the degree of substitution of the functional moiety (DS) is in the range of about 0.01 to about 1.5, such as about 0.05 to about 1.2, such as about 0.1 to about 1, such as about 0.2 to about 0.8, such as about 0.4 to about 0.6.

The amount of substitution may also be quantified as equivalents of charge per base unit mass of cellulose (meq/g). For positively charged functional moieties, such as QA and DEAE, it has been found that a range of 0.5 to 3.5 meq/g is advantageous.

Thus, an embodiment of the present invention relates to the method as described herein, wherein the functional moiety is QA and/or DEAE, and wherein the equivalents of charge per base unit mass of cellulose on the nanofibrous cellulose scaffold is in the range of about 0.5 meq/g to about 3.5 meq/g, such as 1 meq/g to about 2 meq/g, preferably in the range of about 1.25 meq/g to about 1.75 meq/g.

The functional moieties attached to the cellulose nanofibers may be of either chemical or biological origin. In particular, positively charged groups are advantageous as they induce electrostatic interactions between the nanofibrous cellulose scaffold and cells with a negatively charged membrane, thereby increasing attachment of cells to the microcarrier. Biological moieties include proteins and peptides that are normally an integral part of the interaction between the cell and the extracellular environment. This interaction may further promote attachment of cells to the nanofibrous cellulose scaffold.

Therefore, an embodiment of the present invention relates to the method as described herein, wherein the functional moiety is selected from chemical moieties or biological molecules.

Another embodiment of the present invention relates to the method as described herein, wherein the chemical moieties are selected from the group consisting of negatively charged groups, positively charged groups, zwitterionic groups, hydrophobic groups, hydrophilic groups, amphiphilic groups, ligands, and combinations thereof.

A further embodiment of the present invention relates to the method as described herein, wherein the chemical moieties are selected from the group consisting of quaternary ammonium (QA), carboxymethyl (CM), diethylaminoethyl (DEAE), 4-(1-bromoethyl)benzoic acid, 4-(dimethylamino)pyridine, epoxide, and combinations thereof.

A preferred embodiment of the present invention relates to the method as described herein, wherein the chemical moiety is quaternary ammonium (QA).

It is to be understood that the chemical moieties may be attached to the cellulose backbone using conventional chemistry. Thus reagents such as, but not limited to, 3-chloro-2-hydroxypropyl trimethyl ammonium chloride (CHPTAC), 2-chloro-N,N diethylethylamine hydrochloride (DAECH), and monochloro acetic acid (MCAA) may be used for attachment of QA, DEAE, and CM, respectively.

An even further embodiment of the present invention relates to the method as described herein, wherein the biological molecules are selected from the group consisting of proteins, peptides, antibodies, amino acids, polypeptides, glycoproteins, lipoproteins, and antigens, and combinations thereof.

After functionalization of the processed cellulose nanofiber scaffold, the material is dried to yield the nanofibrous cellulose scaffold in its final shape. Drying may be performed in two steps, such as freezing followed by lyophilization, or in a single step, such as by lyophilization. Lyophilizers work by freezing the material, then reducing the pressure and adding heat to allow the frozen water in the material to sublimate. If the water that keeps the processed cellulose nanofibers in solution would be air dried or heat dried, then the slow evaporation of water could cause the final nanofibrous cellulose scaffold collapse, thereby yielding a non-uniform and deformed material. Accordingly, lyophilization is preferred.

Thus, an embodiment of the present invention relates to the method as described herein, wherein the drying step (iv) comprises freezing and/or lyophilization of the processed cellulose nanofiber material.

Another embodiment of the present invention relates to the method as described herein, wherein the drying step (iv) comprises freezing of processed cellulose nanofiber material followed by lyophilization of the frozen processed cellulose nanofiber material.

Drying may be followed by a curing step in which the nanofibrous scaffold is cured to stabilise the crosslinking and improve the mechanical stability. Curing speeds up the process with which the crosslinking takes place.

Therefore, an embodiment of the present invention relates to the method as described herein, wherein the drying step (iv) is followed by a step comprising curing of the dried processed cellulose nanofiber material.

Another embodiment of the present invention relates to the method as described herein, wherein said curing is performed at a temperature in the range of about 20° C. to about 200° C., preferably about 100° C. to about 150° C.

A further embodiment of the present invention relates to the method as described herein, wherein said curing is performed for a period of 2-4 hours.

A still further embodiment of the present invention relates to the method as described herein, wherein said curing is performed at 120° C. for 3 hours.

In one variant of the method, the drying step may be followed by a compression step to reduce the thickness of the material. After the compression step, the nanofibrous cellulose scaffold is cured as described above. By including a compression step, the production of thinner materials is facilitated as the challenge of overcoming surface tension to cover the surface of the mould with a very thin liquid layer is overcome, i.e. the nanofibrous cellulose scaffold may initially be moulded at a greater thickness and by compression be reduced in thickness. This variant of the method may be used to produce e.g. thin discs of the nanofibrous cellulose scaffold.

Thus, an embodiment of the present invention relates to the method as described herein, wherein the curing step is preceded by a compression step comprising compression of the nanofibrous cellulose scaffold to reduce the thickness of the nanofibrous cellulose scaffold.

When the nanofibrous cellulose scaffold is crosslinked and dried, the cellulose nanofibers are locked into a specific shape. The shape will depend on the container in which the solution of processed cellulose nanofiber material is held at the step of drying. In principle, a large batch of the nanofibrous cellulose scaffold can be produced by choosing a large container, and then the final dimensions/shape of the nanofibrous cellulose scaffold can be determined by cutting or punching out the desired shape.

Therefore, an embodiment of the present invention relates to the method as described herein, wherein the geometrical shape of the sample is modified after the drying step (iv).

However, it may be more convenient and efficient to directly dry the processed cellulose nanofiber material in a moulding container of the desired shape. The moulding container could be a cell culturing device as such, or a mould that produces a shape of nanofibrous cellulose scaffold that can be inserted into a cell culturing device.

Thus, a preferred embodiment of the present invention relates to the method as described herein, wherein the processed cellulose nanofiber material is transferred to a moulding container prior to the drying step (iv).

Another embodiment of the present invention relates to the method as described herein, wherein the moulding container is selected from the group consisting of a mould, a cell culturing plate, a bioreactor, a cell culture bottle, a spinner flask, a shaker flask, and a roller bottle, a petri dish, and a tube, preferably a mould.

Yet another embodiment of the present invention relates to the method as described herein, wherein the mould is shaped to provide a casting of the processed cellulose nanofiber material in a geometry selected from the group consisting of a cylinder, disk, cube, sheet, and sphere.

In particular, it is preferred to mould the processed cellulose nanofiber material to a disc suitable for use in a bioreactor.

Thus, a preferred embodiment of the present invention relates to the method as described herein, wherein the nanofibrous cellulose scaffold is a disc with a thickness in the range of about 3 mm to about 30 mm, such as about 3 mm to about 20 mm, such as about 3 mm to about 15 mm, preferably about 3 mm to about 10 mm.

A further embodiment of the present invention relates to the method as described herein, wherein the moulding container is made from a material selected from the group consisting of metal, plastic, glass, ceramic, and composite, and combinations thereof.

The method described herein provides a nanofibrous cellulose scaffold with large surface area and low dead volume that may advantageously be utilised as a microcarrier for culturing of cells. Because the cellulose nanofibers are crosslinked the nanofibrous cellulose scaffold take the form of a dry continuous material with sufficient mechanical strength for use as a microcarrier in a packed bed bioreactor.

Thus, an aspect of the present invention relates to a nanofibrous cellulose scaffold obtainable from a method as described herein.

Another aspect of the present invention relates to a nanofibrous cellulose scaffold comprising a processed cellulose nanofiber material with a mean length of the cellulose nanofibers of less than about 250 μm, and wherein the processed cellulose nanofiber material is crosslinked with a crosslinking agent.

The cellulose nanofibers are connected by the crosslinking agent in a three-dimensional network that provides mechanical strength to the microcarrier material. The crosslinking agent may be any crosslinking agent as described herein.

Thus, an embodiment of the present invention relates to the nanofibrous cellulose scaffold as described herein, wherein the cellulose nanofibers of the processed cellulose nanofiber material are connected by the crosslinking agent in a three-dimensional network.

By dividing the cellulose nanofibers, a material with greatly enhanced surface area and low dead volume is obtained. Without being bound theory, it is also contemplated that the shorter nanofiber strands may contribute to the mechanical strength of the nanofibrous cellulose scaffold, as shorter fiber strands in general will appear stiffer than their longer counterparts. Functionalization as described herein may further provide the nanofibrous cellulose scaffold with desired properties such as positive charges to induce attachment of cells to the cellulose nanofibers. The functional moieties bound to the cellulose nanofibers may be as described herein.

Thus, an embodiment of the present invention relates to the nanofibrous cellulose scaffold as described herein, wherein the cellulose nanofibers have a mean length in the range of about 30 μm to about 250 μm, such as about 40 μm to about 200 μm, such as about 50 μm to about 150 μm, preferably about 60 μm to about 100 μm.

Favourable crosslinking agent to cellulose ratios have been identified that provide nanofibrous cellulose scaffolds which increase cell proliferation. The selected crosslinking agent to processed cellulose nanofiber material ratios are advantageous in that they result in a microcarrier that allow cells to freely migrate within the porous structure but at the same time provide sufficient mechanical strength for the microcarrier to be suitable for used in stirred bioreactors.

Therefore, an embodiment of the present invention relates to the nanofibrous cellulose scaffold as described herein, wherein the ratio of crosslinking agent to processed cellulose nanofiber material is in the range of about 52*10⁻³ mol %/mol % to about 175*10⁻³ mol %/mol %, such as about 5*10⁻³ mol %/mol % to about 100*10⁻³ mol %/mol %, such as about 10*10⁻³ mol %/mol % to about 80*10⁻³ mol %/mol %, such as about 15*10⁻³ mol %/mol % to about 50*10⁻³ mol %/mol %, such as about 25*10⁻³ mol %/mol % to about 40*10⁻³ mol %/mol %.

A preferred embodiment of the present invention relates to the nanofibrous cellulose scaffold as described herein, wherein the ratio of crosslinking agent to processed cellulose nanofiber material is in the range of about 30*10⁻³ mol %/mol % to about 50*10⁻³ mol %/mol %.

It is to be understood that the mol % of the processed cellulose nanofiber material refers to the mol % per glucose unit in the cellulose material.

Another embodiment of the present invention relates to the nanofibrous cellulose scaffold as described herein, wherein the processed cellulose nanofiber material is functionalized with a functional moiety.

The nanofibrous cellulose scaffold has the form of a dry continuous material, the form of which may be adapted to suit existing cell culturing devices, such as packed bed bioreactors. An advantage of the nanofibrous cellulose scaffold is that any shape and dimension can be produced. This means that thicker discs, cylinders, or sheets of the material can be formed, thereby obviating need of stacking thin discs or sheets directly on top of each other to achieve the desired volume of microcarrier. Stacking of microcarrier entities on top of each other may cause inconsistent culturing since the homogeneity of the microcarrier is lost in the interface between. Overall, the nanofibrous cellulose scaffold may act as a homogeneous microcarrier solution which provides a higher surface area and lower dead volume than existing solutions.

Accordingly, an embodiment of the present invention relates to the nanofibrous cellulose scaffold as described herein, wherein the nanofibrous cellulose scaffold is provided as a dry continuous material.

Another embodiment of the present invention relates to the nanofibrous cellulose scaffold as described herein, wherein the physical form of the nanofibrous cellulose scaffold is a geometry selected from the group consisting of a cylinder, disk, cube, sheet, and sphere.

A further embodiment of the present invention relates to the nanofibrous cellulose scaffold as described herein, wherein the nanofibrous cellulose scaffold has a thickness in all three dimensions of at least about 0.1 mm, such as at least about 0.2 mm, such as at least about 0.3 mm, such as at least about 0.4 mm, such as at least about 0.5 mm.

An even further embodiment of the present invention relates to the nanofibrous cellulose scaffold as described herein, wherein the nanofibrous cellulose scaffold is a disk with a thickness in the range of about 0.2 mm to about 2 mm, such as about 0.3 mm to about 1.5 mm, such as about 0.4 mm to about 1.25 mm, preferably about 0.5 mm to about 1 mm.

Thicker discs can be advantages because they can withstand shear forces in a bioreactor to a larger degree than smaller discs. Furthermore, thick discs provide a voluminous continuous scaffold for the cells to proliferate within without artifacts from spaces at the interface of stacked discs.

Thus, an embodiment of the present invention relates to the nanofibrous cellulose scaffold as described herein, wherein the nanofibrous cellulose scaffold has a thickness in all three dimensions of at least about 3 mm, such as at least about 4 mm, such as at least about 5 mm, such as at least about 6 mm, such as at least about 7 mm, such as at least about 8 mm, such as at least about 9 mm, such as at least about 10 mm.

Another embodiment of the present invention relates to the nanofibrous cellulose scaffold as described herein, wherein the nanofibrous cellulose scaffold is a disc with a thickness in the range of about 3 mm to about 30 mm, such as about 3 mm to about 20 mm, such as about 3 mm to about 15 mm, preferably about 3 mm to about 10 mm.

A preferred embodiment of the present invention relates to the nanofibrous cellulose scaffold as described herein, wherein the nanofibrous cellulose scaffold is a disc with a thickness in the range of about 5 mm to about 9 mm.

Still another embodiment of the present invention relates to the nanofibrous cellulose scaffold as described herein, wherein nanofibrous cellulose scaffold is a disk with a diameter in the range of about 50 mm to about 500 mm, such as about 100 mm to about 300 mm, such as about 150 mm to about 200 mm.

A preferred embodiment of the present invention relates to the nanofibrous cellulose scaffold as described herein, wherein nanofibrous cellulose scaffold is a disc with a diameter in the range of about 40 mm to about 70 mm, such as about 50 mm to about 60 mm, preferably about 57 mm.

The disk could also be provided with a small diameter and packed in the bed in a more random-oriented fashion within the bed.

Thus, an embodiment of the present invention relates to the nanofibrous cellulose scaffold as described herein, wherein nanofibrous cellulose scaffold is a disk with a diameter in the range of about 2 mm to about 50 mm, such as about 2 mm to about 25 mm, such as about 2 mm to about 10 mm.

The flexibility of production method means that any shape of nanofibrous cellulose scaffold can be produced to fit existing or future cell culturing devices. The material is both microporous to allow cell migration and proliferation within the support matrix and resilient enough to withstand the shear forces in a packed bed bioreactor.

Thus, an embodiment of the present invention relates to the nanofibrous cellulose scaffold as described herein, wherein the nanofibrous cellulose scaffold is microporous.

A further embodiment of the present invention relates to the nanofibrous cellulose scaffold as described herein, wherein the nanofibrous scaffold is resilient.

The compressive strength of the nanofibrous scaffold may be modified by adjusting the amount of cellulose nanofibers and crosslinking agent.

Thus, an embodiment of the present invention relates to the nanofibrous cellulose scaffold as described herein, wherein the nanofibrous scaffold has a compressive strength in the range of about 0.1 mm/N to about 30 mm/N.

Another embodiment of the present invention relates to the nanofibrous cellulose scaffold as described herein, wherein the nanofibrous scaffold has a compressive strength in the range of about 10 mm/N to about 40 mm/N, such as about 12 mm/N to about 35 mm/N, such as about 15 mm/N to about 30 mm/N, such as about 15 mm/N to about 22 mm/N.

Yet another embodiment of the present invention relates to the nanofibrous cellulose scaffold as described herein, wherein the compressive strength is measured in accordance with ISO 604:2002—Plastics—Determination of compressive properties.

The nanofibrous cellulose scaffold has a low density. Without being bound by theory, it is contemplated that the low density is caused by the homogenous distribution of processed cellulose nanofibers in the material, which ensures that large entanglements and clusters of nanofibers are avoided. Thus, the nanofibrous cellulose scaffold has a significantly reduced density compared to that of pure cellulose in the form of sheets, which is 1.5 g/cm³. Despite the low density, the nanofibrous cellulose scaffold provides a superior available surface area that is advantageous for culturing of adherent cells.

Therefore, an embodiment of the present invention relates to the nanofibrous cellulose scaffold as described herein, wherein the nanofibrous cellulose scaffold has a density in the range of about 0.0005 g/cm³ to about 0.5 g/cm³, such as about 0.001 g/cm³ to about 0.1 g/cm³, such as about 0.001 g/cm³ to about 0.040 g/cm³.

Another embodiment of the present invention relates to the nanofibrous cellulose scaffold as described herein, wherein the nanofibrous cellulose scaffold has a BET surface area of at least about 40000 cm²/g, such as at least about 50000 cm²/g, such as at least about 55000 cm²/g, such as at least about 60000 cm²/g, such as at least about 70000 cm²/g, such as at least about 80000 cm²/g.

Another embodiment of the present invention relates to the nanofibrous cellulose scaffold as described herein, wherein the nanofibrous cellulose scaffold has a BET surface area of at least about 25000 cm²/g, such as at least about 30000 cm²/g, such as at least about 40000 cm²/g, such as at least about 50000 cm²/g, such as at least about 60000 cm²/g, such as at least about 70000 cm²/g.

Yet another embodiment of the present invention relates to the nanofibrous cellulose scaffold as described herein, wherein BET surface area is measured according to ISO 9277:2022—Determination of the specific surface area of solids by gas adsorption—BET method.

The properties of the nanofibrous cellulose scaffold may be further modified by incorporation of additional types of nanofibers in the cellulose nanofiber material. The additional nanofibers may be any of those natural or synthetic polymers described herein.

Therefore, an embodiment of the present invention relates to the nanofibrous cellulose scaffold as described herein, wherein the nanofibrous scaffold further comprises one or more nanofibers selected from natural polymers or synthetic polymers.

Despite the fact that bacterial, yeast, and insect cell expressions systems are capable of overexpressing recombinant proteins, culturing of mammalian cells remains a cornerstone for biomanufacturing of biologics because of their ability to propagate human viruses, express monoclonal antibodies, and incorporate post-translational modifications, such as glycosylation that are critical to the production of effective biologics. Among the most utilised mammalian cell lines are human embryonic kidney (HEK) 293 cells that have been engineered to produce therapeutic proteins and antibodies at high levels. However, HEK 293 cells and other adherent mammalian cells are highly dependent on a suitable support matrix for cell proliferation and viability. One preferred system for culturing of adherent mammalian cells is a packed bed bioreactor. Thus, microcarriers suitable for use with packed bed bioreactors are important products to continue pushing biomanufacturing to become more cost-effective.

The nanofibrous cellulose scaffold described herein offers all the traits (high surface area, low dead volume, adequate mechanical strength, customizable) that are necessary for use as an efficient microcarrier for packing within a packed bed bioreactor. Importantly, the nanofibrous cellulose scaffold can easily be scaled for industrial use without cost and applicability of the microcarrier when used at large volumes being a hindrance.

Accordingly, an aspect of the present invention relates to a microcarrier comprising a nanofibrous cellulose scaffold as described herein.

Another aspect of the present invention relates to use of a nanofibrous cellulose scaffold as described herein as a microcarrier for cell culturing.

A further aspect of the present invention relates to a cell culturing device comprising a container loaded with a nanofibrous cellulose scaffold or a microcarrier as described herein.

The container of the cell culturing devices is not limited to any particular container as long as it is a suitable vessel for culturing of cells.

Therefore, an embodiment of the present invention relates to the cell culturing device as described herein, wherein the container is selected from the group consisting of a bioreactor, a cell culturing plate, a cell culture bottle, a spinner flask, a shaker flask, and a roller bottle, a petri dish, and a tube, preferably a bioreactor.

Bioreactors are of particular importance to large scale industrial production. They may hold larger volumes than laboratory- or pilot study equipment and are used in upstream processes to expand and scale cell culture for production. A typical process involves initial expansion of cells in a smaller vessel followed by successive expansion into larger culture vessels. When the culture volume and density is optimal, the cells are transferred to a production bioreactor, which offers a controlled microenvironment and nutrient delivery to regulate cell growth and differentiation, improving standardization and reproducibility. The nanofibrous cellulose scaffold described herein is suitable for use at any point in this process and is particularly advantageous at the production scale with packed bed bioreactors where other microcarriers may be too expensive for feasible commercial use or unsuitable due to insufficient mechanical strength.

Thus, an embodiment of the present invention relates to the cell culturing device as described herein, wherein the container is a bioreactor, preferably a packed bed bioreactor.

Packed bed bioreactors come in two main categories. One wherein the bed is stationary within the reactor, this is typically referred to as a fixed bed bioreactor. Another wherein the bed is moving within the reactor, this is also known as a dynamic bed bioreactor or moving bed bioreactor. In the latter type of bioreactor the bed may be connected to one or more shafts that moves within the bioreactor, such as by rotation. The nanofibrous cellulose scaffold may be used with any type of packed bed bioreactor.

Thus, an embodiment of the present invention relates to the cell culturing device as described herein, wherein the bioreactor is a fixed bed bioreactor.

Another embodiment of the present invention relates to the cell culturing device as described herein, wherein the bioreactor is a dynamic bed bioreactor.

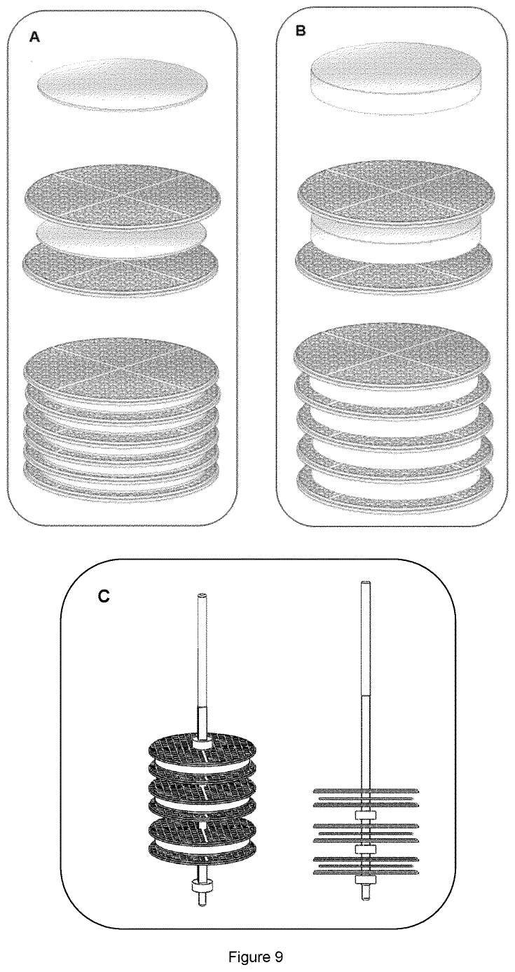

Many of the existing solutions employ stacked discs or sheets of microcarrier material. The stacked discs or sheets are spaced apart by support layers (see FIG. 9A). The support layers may be of plastic and are macroporous to allow flow medium through the packed bed. The nanofibrous cellulose scaffold described herein may be used with these types of solutions and are advantageous in that the ability to form larger continuous discs or sheets allow more microcarrier material per volume to be packed in the bed. Amongst other this is achieved by less support layers being needed per volume (see FIG. 9B).

Thus, an embodiment of the present invention relates to the cell culturing device as described herein, wherein the container is loaded with a plurality of nanofibrous cellulose scaffold layers.

Another embodiment of the present invention relates to the cell culturing device as described herein, wherein each nanofibrous cellulose scaffold layer is spaced apart by a support layer.

Yet another embodiment of the present invention relates to the cell culturing device as described herein, wherein each nanofibrous cellulose scaffold layer is positioned between two support layers.

A further embodiment of the present invention relates to the cell culturing device as described herein, wherein the support layers are macroporous.

A still further embodiment of the present invention relates to the cell culturing device as described herein, wherein the support layers are made of a material selected from the group consisting of plastic, metal ceramic, and composite, and combinations thereof.

An even further embodiment of the present invention relates to the cell culturing device as described herein, wherein nanofibrous cellulose scaffold layers and support layers are alternately positioned in a stack.

Another embodiment of the present invention relates to the cell culturing device as described herein, wherein the stack is capped/terminated by a support layer in each end.

The stacked layers may be positioned on one more shaft in the bioreactor (see FIG. 9C). In the case of a dynamic bed bioreactor, the shaft may be moving (e.g. by rotation) during culturing.

The geometries and dimensions of the nanofibrous cellulose scaffold may be as described herein. The nanofibrous cellulose scaffold can be produced by the simple and cost-efficient method described herein, which the material inexpensive on a surface area per volume basis compared to commercially available microcarriers. The nanofibrous cellulose scaffold is therefore readily scalable to industrial scale production and may be used with large volume bioreactors.

The nanofibrous cellulose scaffold is suitable for use all the way from pilot scale/product development (1 L to 100 L) to production scale (100 L to 1000 L). If required, the material may be also used as microcarrier in even larger bioreactors.

Thus, an embodiment of the present invention relates to the cell culturing device as described herein, wherein the volume of the container is in the range of about 1 L to about 1000 L, such as about 100 L to about 1000 L.

Thus, an embodiment of the present invention relates to the cell culturing device according to item VV, wherein the volume of the container is at least 10 L, such as at least 50 L, such as at least 100 L, such as at least 200 L, such as at least 500 L, such as at least 1000 L.

The container is not limited to any particular material but may be from e.g. stainless steel or of a disposable material, the latter of which provides flexibility and reduces downtime caused by the need for cleaning and sterilization of the culturing device.

The nanofibrous cellulose scaffold may be utilised as microcarrier in a conventional method for cell culturing. The nanofibrous cellulose scaffold may be provided as part of a cell culturing device comprising a container loaded with the microcarrier. It is contemplated that the nanofibrous cellulose scaffold is suitable for use in culturing of any cell line that may benefit from interaction with a support matrix during culturing. It is to be understood that the nanofibrous cellulose scaffold can be used in any traditional scale up step for cell culturing, i.e. first adding microcarrier and cells to a small container (e.g. a flask), then move the cell population to a larger container, before finally transferring the cell culture on to a bioreactor.

Thus, an aspect of the present invention relates to a method for cell culturing comprising the steps of:
(i) providing a cell culturing device as described herein,
(ii) adding a composition comprising a cell population to the cell culturing device,
(iii) incubating the cell population to provide a proliferated cell population, and
(iv) optionally, extracting the proliferated cell population from the cell culturing device.

An embodiment of the present invention relates to the method for cell culturing as described herein, wherein the composition comprises a solvent.

Another embodiment of the present invention relates to the method for cell culturing as described herein, wherein the solvent comprises a cell culturing medium.

The method for cell culturing is particular advantageous for culturing of adherent cells which grow while adhering to the culture vessel. Ideally, cultured cells are cultured in a manner that reflects the conditions under which they exist in the living organism. Adherent cells are cells which under native conditions adhere to tissues.

Thus, an embodiment of the present invention relates to the method for cell culturing as described herein, wherein the cell population comprises adherent cells.

The method for cell culturing can be utilised for achieving many different end results including, but not limited to, production of vaccines, recombinant therapeutic molecules or stem cells. Other growing fields which may benefit from the advantages of the nanofibrous cellulose scaffold includes cellular agriculture production, such as production of existing agricultural products like milk, and (cultured) meat from cells. Cellular agriculture is considered a means of achieving animal-free agriculture. Accordingly, it is contemplated herein that the nanofibrous cellulose scaffold may be used for cellular agriculture. Depending on the desired outcome, different types of cells and cell lines may be cultured.

Thus, an embodiment of the present invention relates to the method for cell culturing as described herein, wherein the origin of the cell population is selected from the group consisting of mammalian cells, human cells, yeast cells, bacterial cells, insect cells and plant cells, preferably mammalian cells.

Another embodiment of the present invention relates to the method for cell culturing as described herein, wherein the cell population comprises a cell type selected from the group consisting of stem cells, kidney cells, lung cells, liver cells, pancreas cells, heart cells, ovary cells, hybridoma cells, and immortalised cells.

A still further embodiment of the present invention relates to the method for cell culturing as described herein, wherein the cell population comprises a cell type selected from the group consisting of myocytes, myoblasts, and adipocytes.

For the purpose of biomanufacturing, some cell lines are preferred due to characteristics such as ease of handling, ability to propagate human viruses, or implementation of favourable glycosylation patterns. An example hereof is HEK293 cells, which may be used for packaging and amplification of recombinant adenovirus.

Therefore, an embodiment of the present invention relates to the method for cell culturing as described herein, wherein the cell population comprises a cell line selected from the group consisting of HEK293 cells, ARN8 cells, Vero cells, MRC-5 cells, WI-38 cells, CHO cells, BHK-21 cells, Sf9 cells, mesenchymal stem cells, pluripotent stem cells, and MDCK cells.

A further embodiment of the present invention relates to the method for cell culturing as described herein, wherein the cell population comprises stem cells.

Another embodiment of the present invention relates to the method for cell culturing as described herein, wherein the cell population comprises a HEK 293 cell line.

The method for culturing cells is preferably performed with agitation of the cell culture within the container to promote better transport of nutrients and oxygen to the cells. The method for cell culturing may be performed in a packed bed bioreactor. In this type of bioreactor typical means of agitation includes, but is not limited to, stirring by an impeller and/or a circulation pump.

Thus, an embodiment of the present invention relates to the method for cell culturing as described herein, wherein incubation is performed under agitation.

Another embodiment of the present invention relates to the method as described herein, wherein agitation is effected by mechanical agitation, circulation pump, and/or pneumatic means.

A further embodiment of the present invention relates to the method as described herein, wherein agitation comprises stirring.

In packed bed bioreactor particularly the agitation creates a flow of solvent within the bioreactor that continuously supplies the cells attached to the microcarrier within the bed with fresh nutrients and oxygen. The direction of the flow may vary depending on the type of bioreactor.

Thus, an embodiment of the present invention relates to the method as described herein, wherein there is a flow of solvent through the cell culturing device.

Another embodiment of the present invention relates to the method as described herein, wherein the flow of solvent is longitudinal and/or radial.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. This is especially true for the description of the method for preparing the nanofibrous cellulose scaffold and all its features, which may readily be part of the nanofibrous cellulose scaffold per se, or a use or method using the same for cell culturing. Embodiments and features of the present invention are also outlined in the following items.

### Items

X1. A method of preparing a nanofibrous cellulose scaffold, said method comprising the steps of:
(i) providing an initial cellulose nanofiber material,
(ii) dividing said initial cellulose nanofiber material into a processed cellulose nanofiber material,
(iii) crosslinking the processed cellulose nanofiber material, and
(iv) drying the processed cellulose nanofiber material,
   thereby providing the nanofibrous cellulose scaffold.

X2. The method according to item X1, wherein the processed cellulose nanofiber material is crosslinked by addition of a crosslinking agent selected from the group consisting of epichlorohydrin, formaldehyde dimethyl acetate (FDA), maleic anhydride (MAL), 3-glycidoxypropyltrimethoxysilane (GPS), citric acid, and 1,2,3,4-butanetetracarboxylic acid.

X3. The method according to any one of items X1 or X2, wherein the crosslinking agent is a polyamide epichlorohydrin resin.

X4. The method according to any one of items X1-X3, wherein the ratio of crosslinking agent to processed cellulose nanofiber material is in the range of about 1:34 (vol %/wt %) to about 1:1 (vol %/wt %), such as about 1:30 (vol %/wt %) to about 1:5 (vol %/wt %), such as about 1:25 (vol %/wt %) to about 1:10 (vol %/wt %), preferably about 1:20 (vol %/wt %) to about 1:15 (vol %/wt %), more preferably about 1:17 (vol %/wt %).

X5. The method according to any one of items X1-X4, wherein the crosslinking step is performed at a temperature in the range of about 15° C. to about 25° C., preferably at about 20° C.

X6. The method according to any one of items X1-X5, wherein dividing said initial cellulose nanofiber material is achieved by dispersing.

X7. The method according to item X6, wherein dispersing is performed with a high-speed disperser.

X8. The method according to any one of items X6 or X7, wherein dispersing is performed for at least 2 min, such as at least 5 min, such as at least 10 min.

X9. The method according to any one of items X6-X8, wherein dispersing is performed at a speed in the range of about 10000 rpm to about 30000 rpm, such as at about 12000 rpm to about 25000 rpm, such as about 15000 rpm to about 20000 rpm.

X10. The method according to any one of the preceding items, wherein the mean length of the cellulose nanofibers in said processed cellulose nanofiber material is shorter than the mean length of the cellulose nanofibers in said initial cellulose nanofiber material.

X11. The method according to any one of the preceding items, wherein the mean length of the cellulose nanofibers in said processed cellulose nanofiber material is less than 250 μm, such as less than about 200 μm, such as less than about 150 μm, such as less than about 120 μm, such as less than about 100 μm, such as less than about 80 μm.

X12. The method according to any one of the preceding items, wherein the mean length of the cellulose nanofibers in said processed cellulose nanofiber material is in the range of about 30 μm to about 250 μm, such as about 40 μm to about 200 μm, such as about 50 μm to about 150 μm, preferably about 60 μm to about 100 μm.

X13. The method according to any one of the preceding items, wherein the initial cellulose nanofiber material is provided as a liquid sample.

X14. The method according to item X13, wherein the solvent of the liquid sample comprises water and/or ethanol.

X15. The method according to any one of the preceding items, wherein the concentration of cellulose nanofibers in the processed cellulose nanofiber material is about 0.1 wt % to about 10 wt %, such as about 0.15 wt % to about 8 wt %, such as about 0.2 wt % to about 5 wt %, such as about 0.2 wt % to about 3 wt %, such as about 0.25 wt % to about 2 wt %, preferably about 0.3 wt % to about 1 wt %, with respect to the total weight of the liquid sample.

X16. The method according to any one of the preceding items, wherein the mean diameter of the cellulose nanofibers in said initial cellulose nanofiber material and/or processed cellulose nanofiber material is in the range of about 10 nm to about 2000 nm, such as about 50 nm to about 1500 nm, such as about 100 nm to about 1000 nm, preferably about 250 nm to about 750 nm.

X17. The method according to any one of the preceding items, wherein the initial cellulose nanofiber material further comprises one or more nanofibers selected from natural polymers or synthetic polymers.

X18. The method according to item X17, wherein the natural polymers are selected from the group consisting of collagen, silk fibroin, keratin, gelatin and polysaccharides, and combinations thereof.

X19. The method according to any one of items X17 or X18, wherein the synthetic polymers are selected from the group consisting of polycaprolactone (PCL), poly(lactic acid) (PLA), polystyrene, polyurethane (PU), poly(lactic-co-glycolic acid) (PLGA), poly(3-hydroxybutyrate-co-3-hydroxyvalerate) (PHBV), and poly(ethylene-co-vinylacetate) (PEVA), and combinations thereof.

X20. The method according to any one of the preceding items, wherein the initial cellulose nanofiber material is prepared by a process selected from the group consisting of electrospinning, meltblowing, drawing, self-assembly, template synthesis, and thermal-induced phase separation.

X21. The method according to any one of the preceding items further comprising a step of functionalizing the processed cellulose nanofiber strands by addition of a reagent comprising a functional moiety, said further step immediately preceding or immediately following the crosslinking step (iii).

X22. The method according to item X21, wherein the functional moiety is selected from chemical moieties or biological molecules.

X23. The method according to item X22, wherein the chemical moieties are selected from the group consisting of negatively charged groups, positively charged groups, zwitterionic groups, hydrophobic groups, hydrophilic groups, amphiphilic groups, ligands, and combinations thereof.

X24. The method according to any one of items X22 or X23, wherein the chemical moieties are selected from the group consisting of quaternary ammonium (QA), carboxymethyl (CM), diethylaminoethyl (DEAE), 4-(1-bromoethyl)benzoic acid, 4-(dimethylamino)pyridine, epoxide, and combinations thereof.

X25. The method according to any one of items X22-X24, wherein the chemical moiety is quaternary ammonium (QA).

X26. The method according to any one of items X22-X25, wherein the biological molecules are selected from the group consisting of proteins, peptides, antibodies, amino acids, polypeptides, glycoproteins, lipoproteins, and antigens, and combinations thereof.

X27. The method according to any one of items X21-X26, wherein the degree of substitution of the functional moiety (DS) is in the range of about 0.01 to about 2.

X28. The method according to any one of items X21-X27, wherein said step of functionalization is preceded by a step of mercerization of said processed cellulose nanofiber material.

X29. The method according to item X28, wherein the mercerization step is immediately before or after the dividing step (ii).

X30. The method according to any one of items X28 or X29, wherein said mercerization step comprises addition of NaOH.

X31. The method according to item X30, wherein concentration of NaOH is in the range of about 0.05 M to about 2 M, such as about 0.1 M to about 1.5 M, such as about 0.25 M to about 0.75 M, preferably about 0.4 M to about 0.6 M.

X32. The method according to any one of items X28-X31, wherein the mercerization step is performed for a period of about 1 hour to about 3 hours, preferably about 2 hours.

X33. The method according to any one of the preceding items, wherein the dividing step (ii) is followed by a step comprising filtration, washing and suspending the processed cellulose nanofiber material.

X34. The method according to item X33, wherein said filtration comprises sieving of the processed cellulose nanofiber material.

X35. The method according to any one of items X33 or X34, wherein the suspension comprises water and/or ethanol.

X36. The method according to any one of the preceding items, wherein the drying step (iv) comprises freezing and/or lyophilization of the processed cellulose nanofiber material.

X37. The method according to any one of the preceding items, wherein the drying step (iv) comprises freezing of processed cellulose nanofiber material followed by lyophilization of the frozen processed cellulose nanofiber material.

X38. The method according to any one of the preceding items, wherein the drying step (iv) is followed by a step comprising curing of the dried processed cellulose nanofiber material.

X39. The method according to item X38, wherein said curing is performed at a temperature in the range of about 20° C. to about 200° C., preferably about 100° C. to about 150° C.

X40. The method according to any one of items X38 or X39, wherein said curing is performed for a period of 2-4 hours.

X41. The method according to any one of items X38-X40, wherein said curing is performed at 120° C. for 3 hours.

X42. The method according to any one of the preceding items, wherein the processed cellulose nanofiber material is transferred to a moulding container prior to the drying step (iv).

X43. The method according to item X42, wherein the moulding container is selected from the group consisting of a mould, a cell culturing plate, a bioreactor, a cell culture bottle, a spinner flask, a shaker flask, and a roller bottle, a petri dish, and a tube, preferably a mould.

X44. The method according to item X43, wherein the mould is shaped to provide a casting of the processed cellulose nanofiber material in a geometry selected from the group consisting of a cylinder, disk, cube, sheet, and sphere.

X45. The method according to any one of items X42-X44, wherein the moulding container is made from a material selected from the group consisting of metal, plastic, glass, ceramic, and composite, and combinations thereof.

Z1. A nanofibrous cellulose scaffold obtainable from a method according to items X1-X45.

Y1. A nanofibrous cellulose scaffold comprising a processed cellulose nanofiber material with a mean length of the cellulose nanofibers of less than about 200 μm, and wherein the processed cellulose nanofiber material is crosslinked with a crosslinking agent.

Y2. nanofibrous cellulose scaffold according to item Y1, wherein the cellulose nanofibers of the processed cellulose nanofiber material are connected by the crosslinking agent in a three-dimensional network.

Y3. The nanofibrous cellulose scaffold according to any one of items Y1 or Y2, wherein the crosslinking agent is selected from the group consisting of epichlorohydrin, formaldehyde dimethyl acetate (FDA), maleic anhydride (MAL), 3-glycidoxypropyltrimethoxysilane (GPS), citric acid, and 1,2,3,4-butanetetracarboxylic acid.

Y4. The nanofibrous cellulose scaffold according to any one of items Y1-Y3, wherein the crosslinking agent is a polyamide epichlorohydrin resin.

Y5. The nanofibrous cellulose scaffold according to any one of items Y1-Y4, wherein the ratio of crosslinking agent to processed cellulose nanofiber material is in the range of about 1:34 (vol %/wt %) to about 1:1 (vol %/wt %), such as about 1:30 (vol %/wt %) to about 1:5 (vol %/wt %), such as about 1:25 (vol %/wt %) to about 1:10 (vol %/wt %), preferably about 1:20 (vol %/wt %) to about 1:15 (vol %/wt %), more preferably about 1:17 (vol %/wt %).

Y6. The nanofibrous cellulose scaffold according to any one of items Y1-Y5, wherein the cellulose nanofibers have a mean length in the range of about 30 μm to about 250 μm, such as about 40 μm to about 200 μm, such as about 50 μm to about 150 μm, preferably about 60 μm to about 100 μm.

Y7. The nanofibrous cellulose scaffold according to any one of items Y1-Y6, wherein the cellulose nanofibers have a mean diameter in the range of about 10 nm to about 2000 nm, such as about 50 nm to about 1500 nm, such as about 100 nm to about 1000 nm, preferably about 250 nm to about 750 nm.

Y8. The nanofibrous cellulose scaffold according to any one of items Y1-Y7, wherein the cellulose nanofiber material is electrospun, meltblown or drawn, preferably electrospun.

Y9. The nanofibrous cellulose scaffold according to any one of items Y1-Y8, wherein the processed cellulose nanofiber material is functionalized with a functional moiety.

Y10. The nanofibrous cellulose scaffold according to item Y9, wherein the functional moiety is selected from chemical moieties or biological molecules.

Y11. The nanofibrous cellulose scaffold according to item Y10, wherein the chemical moieties are selected from the group consisting of negatively charged groups, positively charged groups, zwitterionic groups, hydrophobic groups, hydrophilic groups, amphiphilic groups, ligands, and combinations thereof.

Y12. The nanofibrous cellulose scaffold according to any one of items Y10 or Y11, wherein the chemical moieties are selected from the group consisting of quaternary ammonium (QA), carboxymethyl (CM), diethylaminoethyl (DEAE), 4-(1-bromoethyl)benzoic acid, 4-(dimethylamino)pyridine, epoxide, and combinations thereof.

Y13. The nanofibrous cellulose scaffold according to any one of items Y10-Y12, wherein the chemical moiety is quaternary ammonium (QA).

Y14. The nanofibrous cellulose scaffold according to any one of items Y10-Y13, wherein the biological molecules are selected from the group consisting of proteins, peptides, antibodies, amino acids, polypeptides, glycoproteins, lipoproteins, and antigens, and combinations thereof.

Y15. The nanofibrous cellulose scaffold according to any one of items Y10-Y14, wherein the degree of substitution of the functional moiety (DS) is in the range of about 0.01 to about 2.

Y16. The nanofibrous cellulose scaffold according to any one of items Y1-Y15, wherein the nanofibrous cellulose scaffold is provided as a dry continuous material.

Y17. The nanofibrous cellulose scaffold according to any one of items Y1-Y16, wherein the physical form of the nanofibrous cellulose scaffold is a geometry selected from the group consisting of a cylinder, disk, cube, sheet, and sphere.

Y18. The nanofibrous cellulose scaffold according to any one of items Y1-Y17, wherein the nanofibrous cellulose scaffold has a thickness in all three dimensions of at least about 0.1 mm, such as at least about 0.2 mm, such as at least about 0.3 mm, such as at least about 0.4 mm, such as at least about 0.5 mm.

Y19. The nanofibrous cellulose scaffold according to any one of items Y1-Y18, wherein nanofibrous cellulose scaffold is a disk with a diameter in the range of about 50 mm to about 500 mm, such as about 100 mm to about 300 mm, such as about 150 mm to about 200 mm.

Y20. The nanofibrous cellulose scaffold according to any one of items Y1-Y19, wherein the nanofibrous scaffold is provided as a lyophilized material.

Y21. The nanofibrous cellulose scaffold according to any one of items Y1-Y20, wherein the nanofibrous scaffold is resilient.

Y22. The nanofibrous cellulose scaffold according to any one of items Y1-Y21, wherein the nanofibrous cellulose scaffold has a BET surface area of at least about 40000 cm²/g, such as at least about 50000 cm²/g, such as at least about 55000 cm²/g, such as at least about 60000 cm²/g, such as at least about 70000 cm²/g, such as at least about 80000 cm²/g.

Y23. The nanofibrous cellulose scaffold according to any one of items Y1-Y22, wherein the nanofibrous cellulose scaffold is microporous.

Y24. The nanofibrous cellulose scaffold according to any one of items Y1-Y23, wherein the nanofibrous cellulose scaffold has a density in the range of about 0.0005 g/cm³ to about 0.5 g/cm³, such as about 0.001 g/cm³ to about 0.1 g/cm³, such as about 0.001 g/cm³ to about 0.040 g/cm³.

Y25. The nanofibrous cellulose scaffold according to any one of items Y1-Y24, wherein the nanofibrous scaffold has a compressive strength in the range of about 0.1 mm/N to about 30 mm/N.

Y26. The nanofibrous cellulose scaffold according to any one of items Y1-Y25, wherein the nanofibrous scaffold further comprises one or more nanofibers selected from natural polymers or synthetic polymers.

Y27. The nanofibrous cellulose scaffold according to item Y26, wherein the natural polymers are selected from the group consisting of collagen, silk fibroin, keratin, gelatin and polysaccharides, and combinations thereof.

Y28. The nanofibrous cellulose scaffold according to any one of items Y26 or Y27, wherein the synthetic polymers are selected from the group consisting of polycaprolactone (PCL), poly(lactic acid) (PLA), polystyrene, polyurethane (PU), poly(lactic-co-glycolic acid) (PLGA), poly(3-hydroxybutyrate-co-3-hydroxyvalerate) (PHBV), and poly(ethylene-co-vinylacetate) (PEVA).

A1. A microcarrier comprising a nanofibrous cellulose scaffold according to items Y1-Y28 or Z1.

U1. Use of a nanofibrous cellulose scaffold according to items Y1-Y28 or Z1 as a microcarrier for cell culturing.

V1. A cell culturing device comprising a container loaded with a nanofibrous cellulose scaffold according to any one of items Y1-Y28 or Z1 or a microcarrier according to item A1.

V2. The cell culturing device according to item V1, wherein the container is selected from the group consisting of a bioreactor, a cell culturing plate, a cell culture bottle, a spinner flask, a shaker flask, and a roller bottle, a petri dish, and a tube, preferably a bioreactor.

V3. The cell culturing device according to any one of items V1 or V2, wherein the container is a bioreactor, preferably a packed bed bioreactor.

V4. The cell culturing device according to any one of items V2 or V3, wherein the bioreactor is a dynamic bed bioreactor.

V5. The cell culturing device according to any one of items V1-V4, wherein the volume of the container is at least 10 L, such as at least 50 L, such as at least 100 L, such as at least 200 L, such as at least 500 L, such as at least 1000 L.

V6. The cell culturing device according to any one of items V1-V5, wherein the container is loaded with a plurality of nanofibrous cellulose scaffold layers.

V7. The cell culturing device according to item V6, wherein each nanofibrous cellulose scaffold layer is spaced apart by a support layer.

V8. The cell culturing device according to any one of items V6 or V7, wherein the support layers are macroporous.

V9. The cell culturing device according to any one of items V6-V8, wherein the support layers are made of a material selected from the group consisting of plastic, metal ceramic, and composite, and combinations thereof.

V10. The cell culturing device according to any one of items V6-V9, wherein nanofibrous cellulose scaffold layers and support layers are alternately positioned in a stack.

V11. The cell culturing device according to item V10, wherein the stack is capped/terminated by a support layer in each end.

V12. The cell culturing device according to any one of items V6-V11, wherein the physical form of the nanofibrous cellulose scaffold layer is a geometry selected from the group consisting of a cylinder, disk, cube, sheet, and sphere, preferably a disk.

V13. The cell culturing device according to any one of items V1-V12, wherein the nanofibrous cellulose scaffold layer has a thickness in all three dimensions of at least about 0.1 mm, such as at least about 0.2 mm, such as at least about 0.3 mm, such as at least about 0.4 mm, such as at least about 0.5 mm.

V14. The cell culturing device according to any one of items V1-V13, wherein nanofibrous cellulose scaffold layer is a disk with a diameter in the range of about 25 mm to about 500 mm, such as about 50 mm to about 400 mm, such as about 100 mm to about 300 mm, such as about 150 mm to about 200 mm.

V15. The cell culturing device according to any one of items V1-V14, wherein the cell culturing device comprises a solvent.

V16. The cell culturing device according to item V15, wherein the solvent is cell culturing medium.

T1. A method for cell culturing comprising the steps of:
(i) providing a cell culturing device according to any one of items V1-V16,
(ii) adding a composition comprising a cell population to the cell culturing device,
(iii) incubating the cell population to provide a proliferated cell population, and
(iv) optionally, extracting the proliferated cell population from the cell culturing device.

T2. The method according to item T1, wherein the composition comprises a solvent.

T3. The method according to item T2, wherein the solvent comprises a cell culturing medium.

T4. The method according to any one of items T1-T3, wherein the cell population comprises adherent cells.

T5. The method according to any one of items T1-T4, wherein the origin of the cell population is selected from the group consisting of mammalian cells, human cells, yeast cells, bacterial cells, insect cells and plant cells, preferably mammalian cells.

T6. The method according to any one of items T1-T5, wherein the cell population comprises a cell type selected from the group consisting of stem cells, kidney cells, lung cells, liver cells, pancreas cells, heart cells, ovary cells, hybridoma cells, and immortalised cells.

T7. The method according to any one of items T1-T6, wherein the cell population comprises a cell line selected from the group consisting of HEK293 cells, ARN8 cells, Vero cells, MRC-5 cells, WI-38 cells, CHO cells, BHK-21 cells, Sf9 cells, mesenchymal stem cells, pluripotent stem cells, and MDCK cells.

T8. The method according to any one of items T1-T7, wherein incubation is performed under agitation.

T9. The method according to item T8, wherein agitation is effected by mechanical agitation, circulation pump, and/or pneumatic means.

T10. The method according to any one of items T8 or T9, wherein agitation comprises stirring.

T11. The method according to any one of items T1-T10, wherein there is a flow of solvent through the cell culturing device.

T12. The method according to item T11, wherein the flow of solvent is longitudinal and/or radial.

The invention will now be described in further details in the following non-limiting examples.

### EXAMPLES

### Example 1: Preparation of Nanofibrous Cellulose Scaffold

In this example is given a non-limiting demonstration of how the nanofibrous cellulose scaffold can be prepared. The core properties of the material, such as surface area, degree of substitution, and compression profile, were characterized.

### Method

### Preparation of Initial Cellulose Nanofiber Material

A solution of 19% cellulose acetate was prepared by addition of cellulose acetate (6.38 g) to 12 ml of acetone in an Erlenmeyer flask, followed by addition of 12 ml DMF and 6 ml 96% ethanol. The solution was stirred using a magnetic stirrer overnight at room temperature.

The 19% cellulose acetate solution was electrospun to prepare cellulose acetate nanofiber sheets. The cellulose nanofibers were electrospun (Fluidnatek LE50) using a drum speed of 200 rpm and a flow of 10 ml/hour at 18 kV+ (emitter) and 10 kV− (collector). The cellulose acetate nanofibers were collected on an aluminium substrate, with temperature being 23° C. and at a relative humidity of 63%. After the electrospinning process completed, the cellulose acetate sheet comprising cellulose nanofibers was removed from the drum.

The cellulose acetate sheets were regenerated to cellulose by submergence in a 0.5 M NaOH solution in ethanol. The cellulose acetate sheets were left in the solution for 6 hours followed by transfer to a sieve and washing with copious volumes of distilled water. The washed sheets were put in the oven at 80° C. for 12 hours to provide dry cellulose sheets. The cellulose sheets were weighed.

### Processing of Initial Cellulose Nanofiber Material

The dry cellulose sheets were cut into rough pieces of approximately 2×2 cm squares using scissors. The size of the pieces does not have to be exact, but larger pieces should be avoided as they may hamper the dividing step. The rough pieces of cellulose were fully submerged in water and dispersed using a high-speed disperser (IKA T25 digital Ultra Turrax) at 18,000 rpm for 60 min (with intermittent stops to cool the disperser). The processed cellulose nanofiber material was transferred to a sieve, washed with water to remove any remaining acetate ions, and drained to remove excess water. Cellulose nanofiber material was transferred to a flask and fresh water was added to provide a cellulose concentration of 2 wt %.

Samples with a cellulose concentration of 0.33 wt %, 0.5 wt %, 0.66 wt %, and 1.5 wt % were also prepared. The following steps are described for 2 wt % cellulose, but equally applies to other cellulose concentrations.

### Functionalization of Processed Cellulose Nanofiber Material

The processed cellulose nanofiber material was functionalized with different chemical moieties according to the processes below.

### Quaternary Ammonium (QA):

0.4 g NaOH was dissolved in 20 ml water. 0.4 g regenerated cellulose nanofiber material was then added to the solution. Mercerization continued for 2 hours at room temperature.

The temperature of the cellulose suspension was increased to 80° C. and 3.4 ml of 60% 3-chloro-2-hydroxypropyltrimethyl ammonium chloride (CHPTAC) was added dropwise. The reaction was continued at 80° C. for 4 hours. Then the reaction mixture was filtered after cooling down to room temperature and washed repeatedly with water to remove any unreacted CHPTAC or NaOH. Finally, the filtered processed and functionalized cellulose nanofiber material was mixed with water to make a 2% suspension (or 0.5% or 1.5%) again for further experimentation.

This functionalization protocol together with crosslinking yielded a nanofibrous cellulose scaffold termed “Sponge QA”.

### Carboxymethyl (CM):

0.6 g NaOH was dissolved in 25 ml ethanol. 0.5 g regenerated cellulose nanofiber material was then added to the solution. Mercerization was continued for 1 hour at room temperature.

The temperature of the cellulose suspension was increased to 60° C. and 0.14 g monochloro acetic acid (MCAA) dissolved in 1.2 ml ethanol was added dropwise. The reaction was continued at 60° C. for 2 hours. Filtration and washing were performed as for QA functionalization.

This functionalization protocol together with crosslinking yielded a nanofibrous cellulose scaffold termed “Sponge CMC”.

### Crosslinking of Processed Cellulose Nanofiber Material

The processed cellulose nanofiber material was subjected to crosslinking using polyamide epichlorohydrin (Kymene GHP20 (Solenis), hereafter “Kymene”) as a crosslinking agent. The processed cellulose nanofiber material was poured into distilled water and a calculated amount of Kymene was added dropwise and mixed mechanically for 45 s at 18000 rpm to make suspensions of varying Kymene to cellulose ratios (1:34, 1:17, 1:10, 1:5, and 1:2 vol %/wt % Kymene:cellulose). The resultant suspensions were poured into moulds of desired dimensions. Examples of dimensions produced include a height of 60 mm, 30 mm or 10 mm and inner diameter of 19 mm.

### Drying

Moulds containing the cellulose nanofiber material suspension (crosslinked material) was transferred to a freezer and left for 24 hours at −84° C. The moulds containing the frozen cellulose nanofiber material was then transferred to a lyophilizer and processed for 24 hours at a pressure of <0.001 bar and a temperature of −84° C. to yield a dry continuous material.

### Curing

The dry continuous material was extracted from the mould and cured in the oven for 3 hours at 120° C.

The dry continuous and cured material is also referred to as “sponge” herein.

### Scanning Electron Microscopy

Morphological studies of the nanofibrous cellulose scaffolds were carried out using scanning electron microscopy (SEM). The samples were dried and sputter coated with gold before carrying out the analysis. The micrographs were obtained in secondary electron (SE) imaging mode on a Hitachi SU3500 at an accelerating voltage of 5 kV and a working distance of 7 mm at varying magnifications.

### FTIR Spectroscopy

FTIR analysis was performed on solid dried samples at room temperature using a Perkin-Elmer Spectrum Two instrument provided with Universal ATR in the range of 450-3600 cm⁻¹. The samples were dried at 80° C. for 2 hours before carrying out the analysis.

### Surface Area Measurements

The Brunauer-Emmett-Teller (BET) model was employed to measure the specific surface areas using a molecule of nitrogen bearing molecular cross-sectional area of 0.162 nm². Dried samples were degassed under vacuum for 6 hours before the BET surface area was measured. Nitrogen adsorption and desorption isotherms were acquired on an ASAP 2020 M analyzer (Micromeritics) at 77.3 K.

### Elemental Analysis and Degree of Substitution

Elemental analysis of the nanofibrous cellulose scaffold was performed on an elemental analyzer FlashEA 1112 (Thermo Fischer Scientific) using 5 mg of sample. The samples were well dried before the analysis to remove any adsorbed moisture.

Degree of substitution (DS) of QA functionalized cellulose (i.e. Sponge QA 2%) was calculated using the following formula:

DS=(162N/(1400−cA×N))

where 162 is the molecular weight of the anhydrous glucose unit (AGU); N is the percentage of nitrogen; CA is the molecular weight of the cationic reagent. For the present nanofibrous cellulose scaffold the cationic reagent is CHPTAC with a molecular weight of 188.1.

### Compression Analysis

Compression analysis was performed on a Zwick test machine (inv. No. BX32358) using a 50 N load cell (inv. No. BX32803), a preload of 0,002 N was set and the test speed was 1 mm/min. The nanofibrous cellulose scaffolds were cut to a height of 15 mm before carrying out the analysis.

The compression modulus measures the stiffness of the material or the ability of the material to withstand changes in length when subjected to compressive loads. The higher the compression modulus, the stiffer the material.

### Results

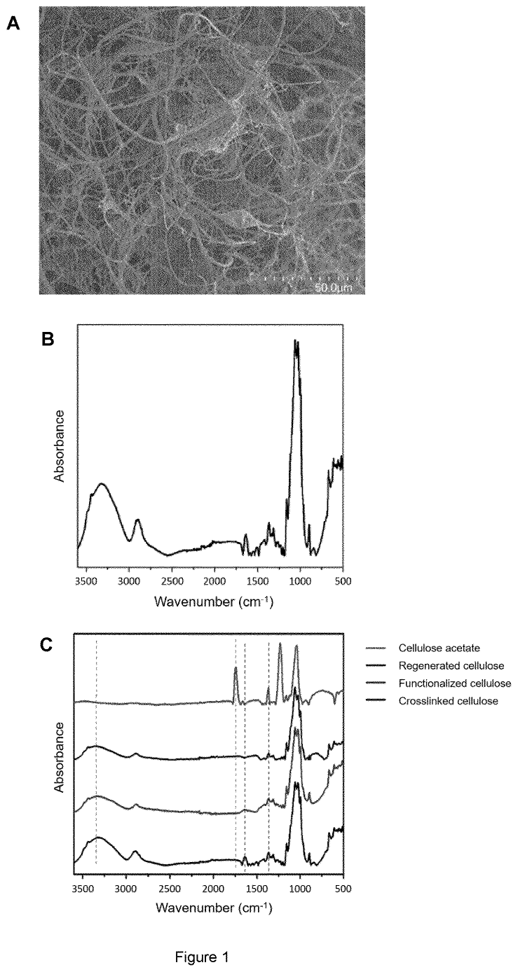

Nanofibrous cellulose scaffolds with a crosslinker and different types of functional moieties were prepared and the dry continuous material was imaged by SEM (FIG. 1A). The resulting nanofibrous cellulose scaffold is highly homogenous in the sense that no large degree of entanglement or clusters of nanofibers are present. The homogenous distribution of nanofibers in the material ensures optimal exposure of the surface area for cell attachment and interaction. The crosslinking agent Kymene could be seen trapped between the cellulose nanofibers (FIG. 1A) adding mechanical strength to the nanofibrous cellulose scaffold.

FTIR analysis was carried out to confirm the formation of linkages in the crosslinked cellulose. The band centered around 1633 cm⁻¹ was attributed to stretching vibrations of carbonyl (C═O) groups incorporated into the cellulosic framework after crosslinking of Kymene (FIG. 1B). A comparison of FTIR spectra of cellulose acetate, regenerated cellulose, QA functionalized cellulose and crosslinked cellulose (FIG. 1C) shows the successful transformation of cellulose acetate to crosslinked cellulose through QA functionalized cellulose. Disappearance of the absorbance band at 1743 cm⁻¹ belonging to the carbonyl group (C═O) of cellulose acetate and appearance of a band at 3345 cm⁻¹ belonging to hydroxyl groups (OH) of cellulose indicated success of regeneration of cellulose acetate to form cellulose nanofibers. Furthermore, the absorbance band at 865 cm⁻¹ associated with quaternary ammonium functionality corroborated the successful formation of QA functionalized cellulose. Appearance of an additional absorbance band at 1633 cm⁻¹ indicated that crosslinking has occurred in the cellulose architecture.

Elemental composition and degree of substitution (DS) was assessed for the nanofibrous cellulose scaffold functionalized with QA (Sponge QA 2%). The content of carbon (C) was 37.71%, the content of hydrogen (H) was 6.59%, and the content of nitrogen (N) was 1.28%. Based on the content of N, the DS was calculated to 0.18. The amount of substituted QA may influence the cell growth as the positive charge facilitates electrostatic interactions with negatively charged cell membranes.

Surface area measurements demonstrated that a surprisingly large BET surface area of 60000 cm²/g was obtained for the nanofibrous cellulose scaffold (Sponge QA 2%). Without being bound by theory, it is contemplated that the short cellulose strands yield a material with increased degree of exposed surface. The large surface area is significantly higher than commercially available microcarriers currently on the market.

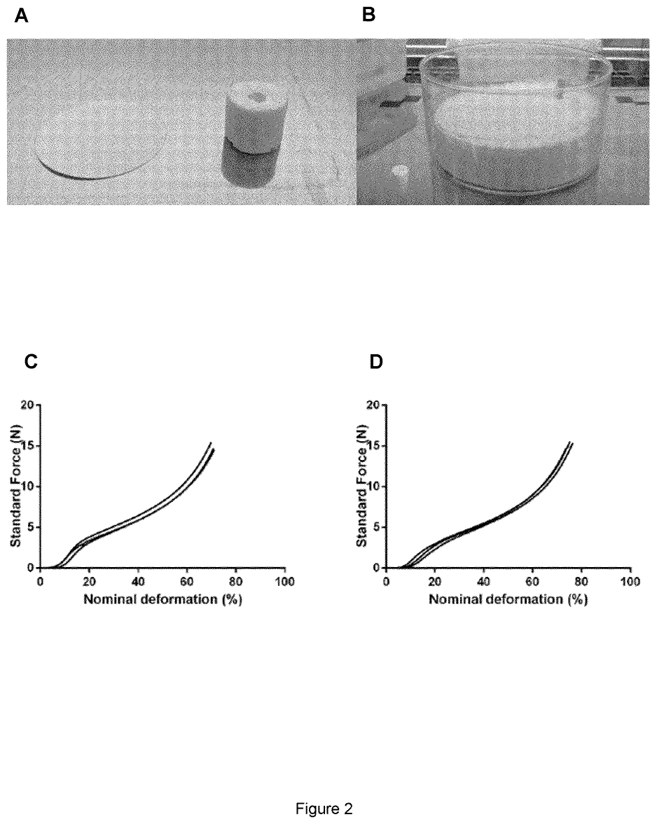

The successful crosslinking of the cellulose nanofibers provided the material with the mechanical strength necessary to yield a dry continuous material that can be moulded and formed to fit the desired application (FIG. 2A-B). This was true for all the nanofibrous cellulose scaffold produced with varying amounts of cellulose and crosslinker.

Compression testing was used to evaluate the behaviour of the nanofibrous cellulose scaffold under a uniaxial compressive load. Change of length (or height), dL, was measured by subjecting the nanofibrous cellulose scaffold to compressive loads and the behaviour of the material was recorded (FIG. 2C-D). The force required to bring in a specific deformation (dL/Fmax) was 0.2 mm/N for the nanofibrous cellulose scaffold (Sponge QA 2%, 1:10 vol %/wt % Kymene to cellulose). In particular the force to obtain 60% deformation for the material was measured to be 9.8 N.

As similar measurement was performed for a with a cellulose concentration of 0.5 wt % (Sponge QA 0.5%, 1:10 vol %/wt % Kymene to cellulose), yielding a dL/Fmax of 0f 17 mm/N and a force to obtain 60% deformation for the material of 0.4 N.

Similar compression profiles data was obtained with nanofibrous cellulose scaffolds with a cellulose to Kymene ratios varying from 34:1 to 2:1.

The physical properties of the nanofibrous cellulose scaffold surprisingly resulted in a versatile material that revert to its original form after being subjected to deformation. This resilient behaviour is advantageous for moulding and adapting the nanofibrous cellulose scaffold to existing and future cell culture platforms.

### Conclusion

This example demonstrates that it is possible to produce several different variants of the nanofibrous cellulose scaffold in a simple manner which is readily scalable for industrial usage. The nanofibrous cellulose scaffold has a large surface area which is available to the cells and does not comprise nanofibers collapsed in clusters or entangled in a fashion that exclude cells from gaining access to the surface.

The resilient material can easily be integrated into all current cell culture solutions from R&D to clinical manufacturing scale, such as packed bed bioreactors allowing customers to stay with the same cell culturing solution throughout their process development (thus reducing risks and costs).

### Example 2: Processing of the Initial Cellulose Nanofiber Material

In this example various methods of dividing the initial cellulose nanofiber material were tested and their influence on the nanofibrous cellulose scaffold was assessed.

### Method

Preparation of the initial cellulose nanofiber material was performed as described in example 1.

### Processing Protocols

Different methods of dividing the initial cellulose nanofiber material were evaluated. Thus, samples were prepared according to the following:

### Mechanical Cutting (by Scissor):

2.5 g Cellulose sheets were cut up in 10×10 mm pieces with a pair of scissors and assayed directly as fragmented cellulose sheets.

### Blending:

2.5 g cellulose sheets were cut up in 20×20 mm pieces with a pair of scissors and added together with 250 ml water to a lab mixer (LB20, Waring Laboratory). The cellulose nanofiber material was processed at 7000 rpm for varying durations of time (0.5, 1, 2.5, 5, and 10 min).

### Laser Cutting:

Cellulose sheets were cut with a laser cutter (Epilog laser, Zing 24) in three different sizes (0.75×0.75 mm, 1.5×1.5 mm and 3×3 mm). The cut sheets were assayed as is.

### Dispersing:

2.5 g cellulose sheets were cut up in 20×20 mm pieces with a pair of scissors and added together with 250 ml water to an Erlenmeyer flask. The sample was processed using a disperser (IKA T25 digital Ultra-Turrax with S25 NB-25 G disperser tool) at 18000 rpm for varying durations of time (0.5, 1, 2.5, 5, 10, 15 and 60 min).

### Microscopy

Samples of nanofibrous cellulose scaffold (diluted to 0.2 wt % cellulose) were added to a glass slide. Images of the network of cellulose nanofibers in the sample were captured on a light microscope (Leica) using 40× magnification.

### Scanning Electron Microscopy (SEM)

Samples of processed cellulose nanofiber material were diluted 1000-10000× in water and a droplet of sample is applied to a SEM fixture. The samples were dried and sputter coated with gold before capturing images on a Hitachi SU3500. Images were captured at different magnifications.

### Fiber Length Measurements

Fiber length of the cellulose nanofibers were determined either by SEM (Hitachi SU3500) or by light scattering (Malvern Mastersizer S).

SEM images were evaluated manually by visual inspection to ensure that all measured fibers had both endings visible. The fiber lengths were determined by use of ImageJ software. Fiber lengths from several SEM images were determined to get a larger dataset.

Fiber lengths were also determined using light scattering. Briefly, 1 ml of sample was added in water to the Malvern Mastersizer S and measurements were performed with the settings described under the definition of “mean length”. Sample was added to the sample container until the obscuration value was between 15-20%. From each sample a fiber length histogram displaying the fiber length distribution was generated. The statistics of the distribution are calculated from the results using the derived diameters D[m,n]—an internationally agreed method of defining the mean and other moments of particle size. D (v, 0.5), D (v, 0.1) and D (v, 0.9) are standard “percentile” readings from the analysis. D (v, 0.5) is the fiber length at which 50% of the sample is smaller and 50% is larger than this length. This value is also known as the Mass median diameter (MMD) when used for particles. D (v, 0.1) is the fiber length for which 10% of the sample is below this length. D (v, 0.9) gives a fiber length for which 90% of the sample is below this length. The volume weighted mean fiber length D[4,3] was also determined.

### Pipetting/Floating Test

Samples of processed cellulose nanofiber material were tested for their ability to be pipetted. 1 ml of sample was pipetted out of the sample container and subsequently expelled from the pipette tip into a tube with water. The ease of pipetting was evaluated, including the propensity for processed cellulose nanofiber material to clog the pipette.

Processed cellulose nanofiber material transferred to a tube containing water was then assessed for its propensity to float. Samples were vigorously shaken and it was observed by visual inspection whether the material floated immediately after and 24 hours after shaking.

### Results

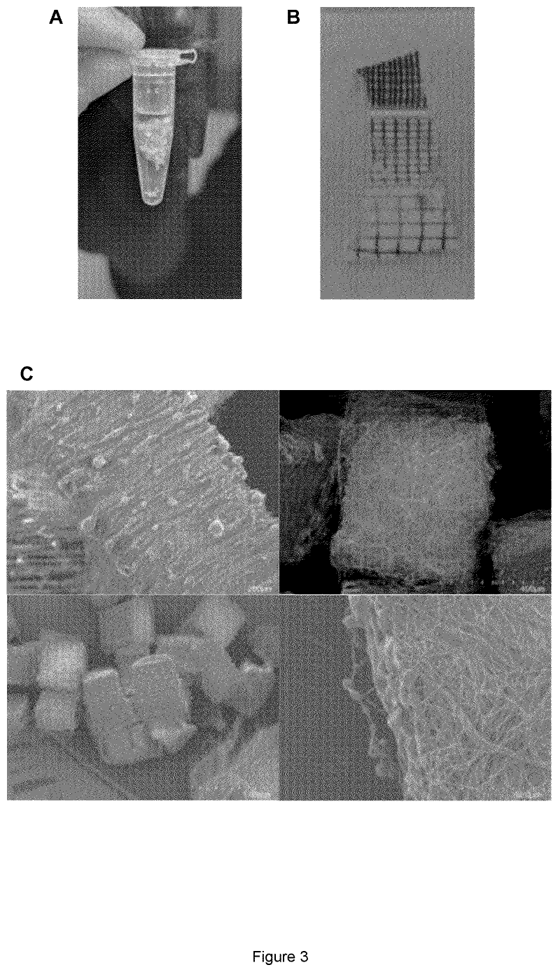

The pipetting test of scissor cut and laser cut cellulose sheets showed that these cut pieces are too large to pipette as they will clog the pipette tip. Also, cellulose nanofiber material processed in this manner generates pieces of dimensions that trap air bubbles and float in solution (FIG. 3A). Furthermore, cutting with a laser makes the cellulose nanofibers melt, burn and stick together and is therefore not a suitable operation for dividing the cellulose nanofibers (FIG. 3B-C).

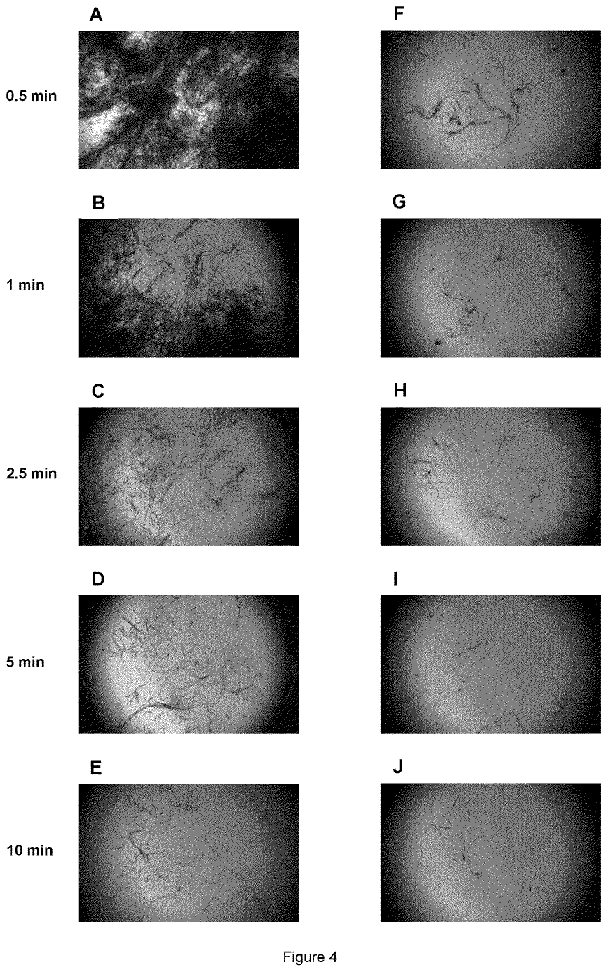

Cellulose nanofiber material processed by the blender (FIG. 4A-E) generally produced more dense and entangled samples with less homogenously distributed cellulose nanofibers than cellulose nanofiber material processed by the disperser (FIG. 4F-J). It appeared that cellulose nanofiber material dispersed for at least 2.5 min was more homogenously distributed throughout the sample and with fewer long cellulose nanofiber strands (FIG. 4F-J). Thus, a minimum of e.g. 2 min of dispersing may be advantageous to avoid excess entanglement and clusters of cellulose nanofibers.

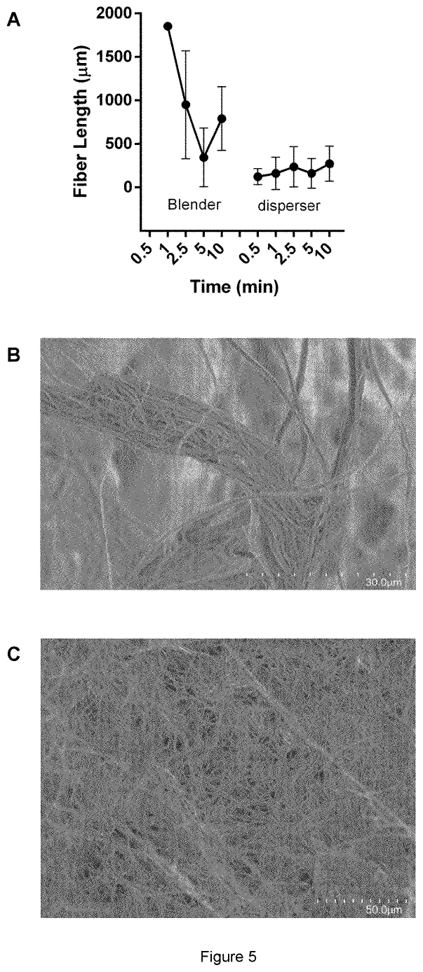

SEM images of the processed cellulose nanofiber material confirmed that the samples processed by blending produced longer (FIG. 5A) and more entangled cellulose nanofibers (FIG. 5B-C) than processed cellulose nanofiber material processed by the dispersing.

The dispersing time influenced the mean length of the cellulose nanofibers in the cellulose nanofiber material. Shorter fiber lengths were obtained for longer dispersing times. Shorter fibers may be advantageous as they are less prone to form cluster and entangle, thereby reducing the amount of non-accessible surface area and the risk of clogging. A summary of the determined mean fiber lengths is given in table 1.

TABLE 1
Fiber length measurements from Malvern Mastersizer S.

| | | | | D[4, 3] |
|---|---|---|---|---|
| | D[v, 0.1] | D[v, 0.5] | D[v, 0.9] | (μm) |
| Dispersing | (μm) | (μm) | (μm) | Volume |
| time | 10% below | 50% below | 90% below | weighted |
| (min) | this value | this value | this value | mean |
| 0.5 | 12 | 195 | 549 | 242 |
| 1 | 9 | 137 | 482 | 198 |
| 2.5 | 6 | 84 | 343 | 135 |
| 5 | 5 | 71 | 306 | 118 |
| 10 | 5 | 64 | 290 | 110 |
| 15 | 5 | 56 | 256 | 97 |
| 60 | 4 | 42 | 192 | 72 |

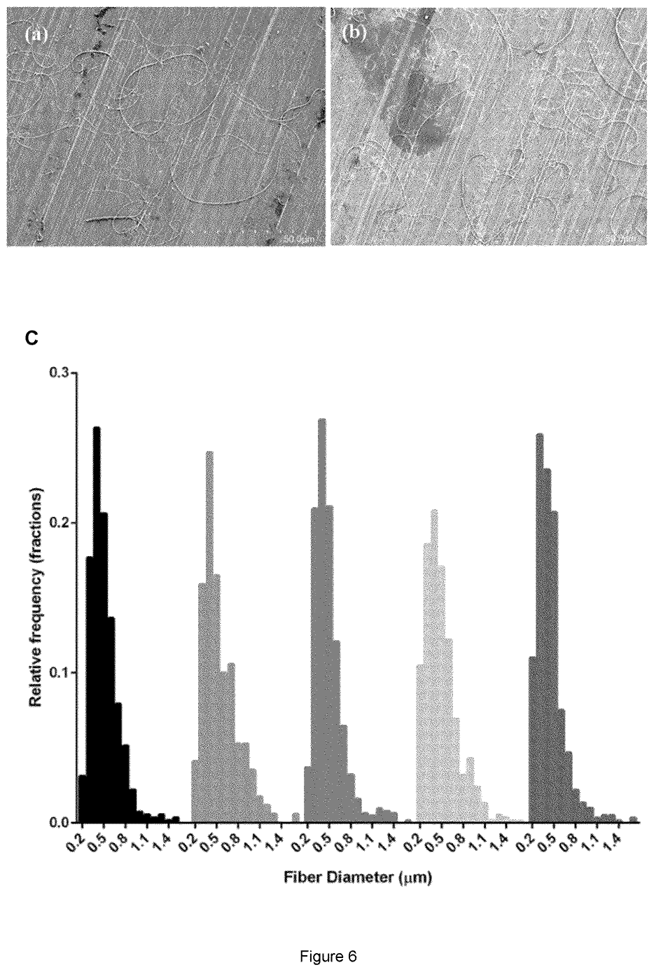

SEM measurements were used to determine cellulose nanofiber diameter (FIG. 6A-B). More than 2000 individual cellulose nanofibers were measured using ImageJ software and gave a mean cellulose nanofiber diameter of 500 nm (FIG. 6C).

None of the cellulose nanofiber materials processed by dispersing trapped air bubbles or had any tendency to float in solution. Pipetting of these samples was least challenging for samples that had been dispersed for longer durations of time. As such it was preferred to disperse for at least 10 min to improve flow through the pipette.

### Conclusion

This example demonstrates that not every method for dividing the initial cellulose nanofiber material is equally effective and suitable for preparation of the nanofibrous cellulose scaffold. In particular, dispersing is advantageous as it homogenously divide the cellulose nanofiber material without generation of entanglements or clusters.

### Example 3: Viability of Cells on the Nanofibrous Cellulose Scaffold

In this example the viability and adherence of cells seeded on the nanofibrous cellulose scaffold was tested.

### Method

### Cell Culturing

HEK293T cells was maintained at 37° C. and 5% CO₂ in 75 cm² culture flasks (T75), the cells were split when they reached 80-90% confluency, approximately every 2-4 days. This was done until the day of seeding the cells onto a microcarrier in a 24-well plate. HEK293T cells were cultured in RPMI-1640 supplemented with 10% FBS and 1% Penicillin/Streptomycin (culture media) throughout all experiments.

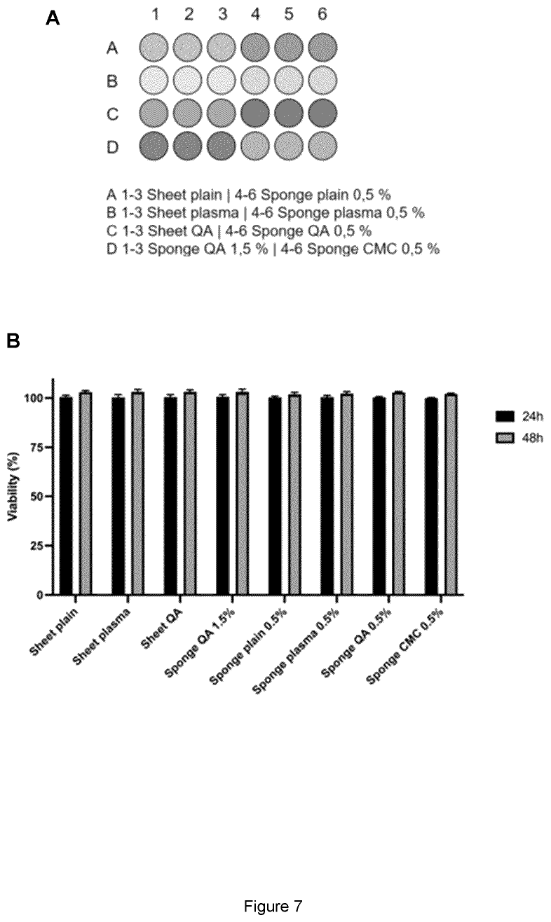

The nanofibrous cellulose scaffold (“sponge”) and a reference cellulose sheet (“sheet”) were tested as microcarriers in the 24-well plate setup according to FIG. 7A. Cellulose sheets were cut manually to fit into the wells. The nanofibrous cellulose scaffold was moulded to fit the wells. The tested microcarrier samples are listed in Table 2.

Plasma treatment was performed by placing the sample in the vacuum chamber (Diener Electronic ATTO Plasma) and adjusting to 0.15 mbar. Oxygen was pumped in to a pressure of 0.25 mbar, and the plasma generator was turned on at 30% power for 5 min. The plasma chamber was aired and the sample was removed.

TABLE 2
Samples for viability testing. Percentages correspond
to the amount of cellulose nanofiber material used in
preparation of the nanofibrous cellulose scaffold.

| Microcarrier | Description |
|---|---|
| Sheet plain | Cellulose sheet, no functionalization |
| Sheet plasma | Cellulose sheet, oxygen plasma treated |
| Sheet QA | Cellulose sheet, functionalized with quaternary |
| | ammonium |
| Sponge plain | Nanofibrous cellulose scaffold, no functionalization, |
| 0.33% | 0.33% cellulose |
| Sponge QA | Nanofibrous cellulose scaffold, functionalized with |
| 0.33% | quaternary ammonium, 0.33% cellulose |
| Sponge CMC | Nanofibrous cellulose scaffold, functionalized with |
| 0.33% | carboxymethyl, 0.33% cellulose |
| Sponge plain | Nanofibrous cellulose scaffold, no functionalization, |
| 0.5% | 0.5% cellulose |
| Sponge plasma | Nanofibrous cellulose scaffold, oxygen plasma treated, |
| 0.5% | |
| Sponge QA | Nanofibrous cellulose scaffold, functionalized with |
| 0.5% | quaternary ammonium, 0.5% cellulose |
| Sponge CMC | Nanofibrous cellulose scaffold, functionalized with |
| 0.5% | carboxymethyl, 0.5% cellulose |
| Sponge plain | Nanofibrous cellulose scaffold, no functionalization, |
| 0.66% | 0.66% cellulose |
| Sponge QA | Nanofibrous cellulose scaffold, functionalized with |
| 0.66% | quaternary ammonium, 0.66% cellulose |
| Sponge CMC | Nanofibrous cellulose scaffold, functionalized with |
| 0.66% | carboxymethyl, 0.66% cellulose |
| Sponge QA | Nanofibrous cellulose scaffold, functionalized with |
| 1.5% | quaternary ammonium, 1.5% cellulose |

500 μl microcarriers were loaded into the wells and preincubated with 1.75 ml PBS for 2 hours at 37° C. Afterwards, the PBS was removed and 750 μl culture media was added to the well. Next, 1×106 HEK293T cells in 100 μl in culture media were seeded onto each microcarrier. Following cell seeding, the 24-well plate was incubated overnight to enable cell attachments to the microcarrier.

Following overnight incubation, 1 ml of culture media was added to each well and the plate was incubated on a shaker (120 rpm) at 37° C. and 5% CO₂. Every morning and afternoon throughout the experiment a half media change was performed to ensure proper glucose/nutrient levels.

After 24 and 48 hours of incubation, each well was analysed for cell adherence and viability using microscopy and a lactate dehydrogenase (LDA) cytotoxicity kit (Abcam). At the 48 hours time point, 25 μl trypan blue (TB) was added to the wells, and the nanofiber inserts where studied from both the top and bottom by microscopy.

### Microscopy

To analyse cell attachment to the microcarriers, each well of the plate was investigated with microscopy (10× magnification). At the 24 hours timepoint the microcarriers were visualized from the bottom without TB. At the 48 hours, the wells were first analysed from the bottom with 25 μl TB added to each well. Next, the microcarrier were dismantled to allow for investigation of the cell adherence from the top of the microcarrier. For all wells and timepoints three images were captured.

### Cell Viability Analysis

To investigate the viability of the cells within the microcarrier, a LDA cytotoxicity kit was used. The viability was determined from the amount of free LDA in the media, since LDA present in the cell is released upon cell death.

Briefly, 10 μl of supernatant from each well was transferred into a flat bottom 96-well plate. Three technical replicates were done per well. Additionally, a live and a dead cell control was used. The live cell control was HEK293T cells cultured in a 12-well culture plate, and the dead cell control was made by adding 10% lysis solution (from the LDA toxicity kit) to a well in the 12-well plate and incubation for 30 min. After the transfer of supernatant 100 μl LDA reaction mix was added to each well of the analysis plate. This plate was incubated for 30 min at RT, before absorbance measurement was done using SpectraMax i3x at 450 nm. The results from the absorbance measurement were converted to the percentage of live cells using the following formula:

$$100 - ( test\_ABS - LiveCTRL\_ABS ) / ( DeadCTRL\_ABS - LiveCTRL\_ABS ) * 100$$

### Results

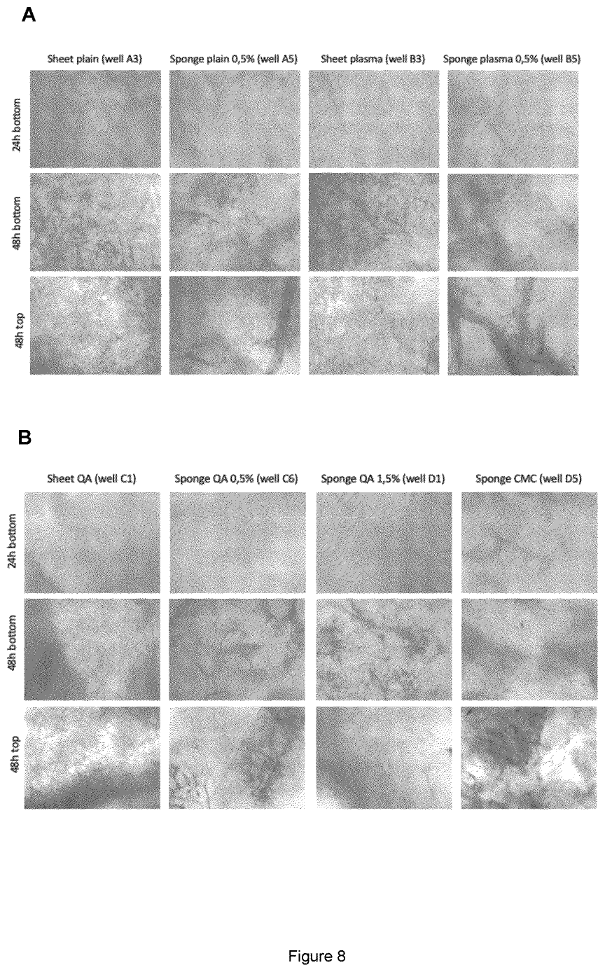

Cells adhered to all microcarrier when investigated under the microscope (FIG. 8A-B). Moreover, no decrease in viability was observed for cells grown on any of the microcarriers (FIG. 7B). In fact, viability was slightly increased (100-105%) at the 48 hours timepoint compared to control live cells cultured without microcarriers. This indicates that the microcarriers improves viability when compared to traditional cell culture in two dimensions. Microcarriers with 0.33 wt % and 0.66 wt % cellulose, respectively, gave similar results, i.e. no decrease in viability (data not shown).

### Conclusion

This example demonstrates that viability is high for cells seeded on the nanofibrous cellulose scaffolds, and that these consequently are suitable for use as microcarriers.

### Example 4: Processing Nanofibers of Material Different from Cellulose

In this example the processing of a variety of nanofibers different from cellulose were assessed with the aim of identifying auxiliary nanofibers suitable for preparation of nanofibrous scaffolds for growing cell cultures on. The electrospun nanofibers were characterized by scanning electron microscopy (SEM) and by visual inspection (following dividing of the nanofibers).

### Method

### Electrospinning

Nanofibers of polycaprolactone (PCL), poly-L-lactic acid (PLA), a mixture of PCL and PLA (PLA/PCL) and cellulose were electrospun. Electrospinning was performed on a Fluidnatek LE50 apparatus as described in Example 1.

PCL fibers were obtained by dissolving 8% Polycaprolactone pellets (Sigma Aldrich, MW 80000), in Chloroform:Methanol, 1:1 solution. Needle to collector distance was set to 20 cm, flow rate to 3 ml/h, and voltage to 18 kV.

PLA fibers were obtained by dissolving 8% PLLA pellets (Goodfellow, MFR: 24), in Chloroform:Methanol, 3:2. Needle to collector distance was set to 24 cm, flow rate to 4.5 ml/h and voltage to 35 kV.

PLA/PCL fibers were obtained by dissolving PLA pellets (Goodfellow, MFR: 65) and PCL pellets (Sigma Aldrich, MW 80000) 1:2 to an 8% polymer solution in Chloroform:Methanol, 3:2. Needle to collector distance was set to 20 cm, flow rate to 3 ml/h and voltage to 18 kV

1 ml of polymer solution was spun for each sheet of fibers.

### Scanning Electron Microscopy

The electrospun material of PCL, PLA, mixed PCL/PLA and cellulose were imaged on a Hitachi SU3500 as described in Example 1. Images of the nanofiber materials were obtained for both undivided and divided nanofibers.

### Processing of PCL, PLA and PCL/PLA Nanofiber Material

Electrospun sheets of PCL, PLA and mixed PLA/PCL were cut into smaller 10×10 mm pieces and mixed with two different mixing tools for 5 minutes. The cut sheets were mixed either with a blender at 7000 rpm (LB20E Laboratory blender, Waring) or with a disperser at 18000 rpm (IKA T25 digital Ultra Turrax).

### Results

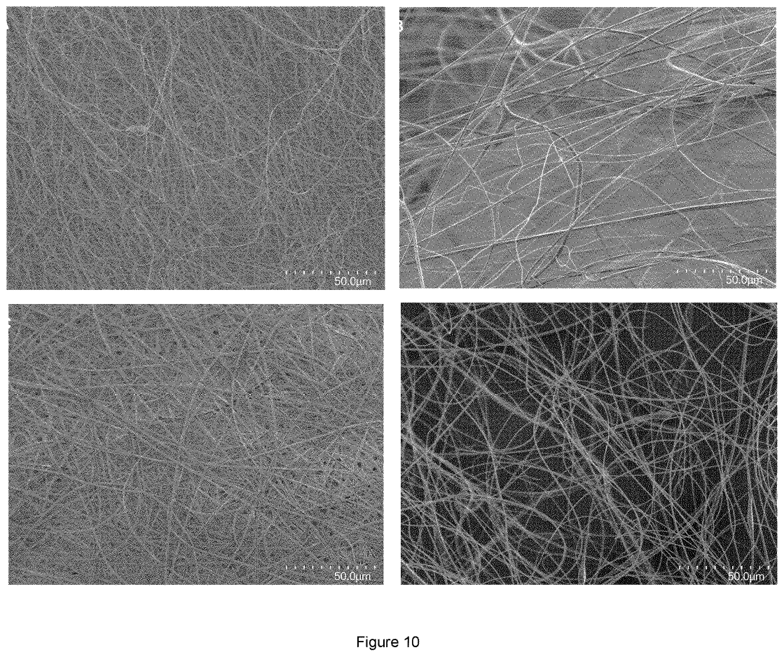

Nanofibers could be electrospun from all materials (PCL, PLA, PLA/PCL, and cellulose) and formed nanofibrous sheets (see FIG. 10A-D).

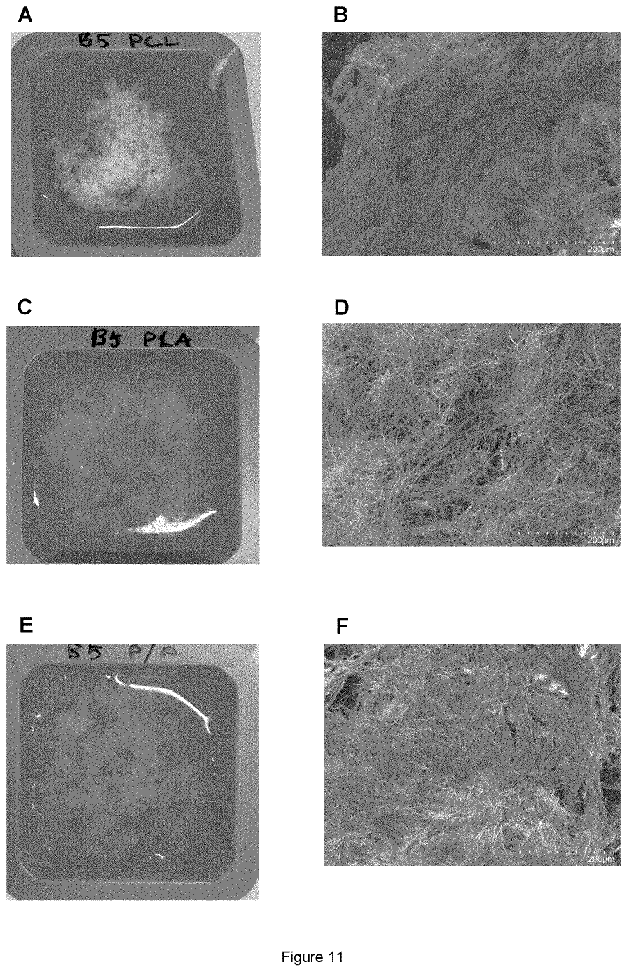

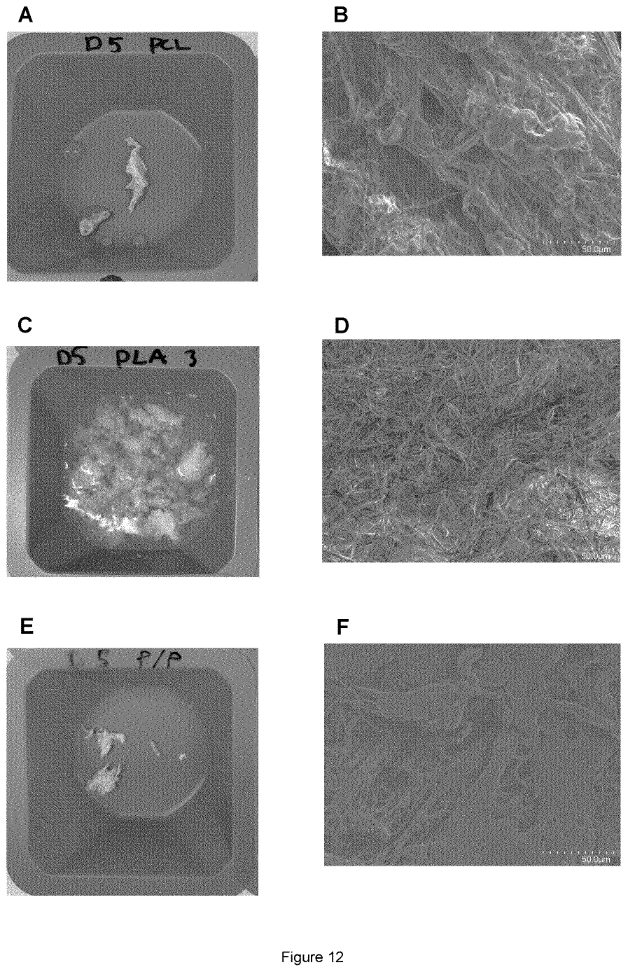

The sheets made from nanofibers different from cellulose were subjected to two individual modes of dividing the nanofibers, namely blending (FIG. 11A-F) and dispersing (FIG. 12A-F). The tests showed that it was not possible to uniformly blend or disperse either PCL, PLA, or PLA/PCL nanofibers into a homogenous mixture of short strand nanofibers.

Both the PCL nanofibers (FIG. 11A-B and FIG. 12A-B) and the PLA/PCL nanofibers (FIG. 11E-F and FIG. 12E-F) melted or deformed during the process. In particular, the nanofibers melted together to form either large pieces of solid polymer or large entangled clusters of nanofibers with semi-melted nanofibers.

The PLA nanofibers (FIG. 11C-D and FIG. 12C-D) did not melt in the same way as the PCL fibers, but all materials did easily get stuck in the mixing tool or got entangled on the blades of the blender and halted the processing.

### Conclusion

This example demonstrates that it is not possible to uniformly blend or disperse nanofibers of all materials. Accordingly, not all nanofiber materials can be easily transformed into shorter strands followed by formation of a nanofibrous scaffold as described herein. Thus, it is preferred to use cellulose nanofibers for the preparation of the nanofibrous scaffolds.

### Example 5: Means of Dividing Initial Cellulose Nanofibers

In this example the influence of the mode of dividing the nanofibers on the length of the cellulose nanofibers was assessed. The nanofibers were divided by either blending or dispersing and the length of representative fractions of nanofibers were measured by individual analysis of SEM images or in solution by laser diffraction.

### Method

QA-functionalized cellulose nanofibers were prepared for further testing. Cellulose acetate sheets were prepared by electrospinning as described in Example 1.

### Regeneration of Cellulose

Cellulose was obtained by regeneration (deacetylation) of electrospun cellulose acetate sheets. Briefly, 25 g cellulose acetate sheets were cut into smaller pieces (2×2 cm) and added to a beaker with 1.25 L of 0.5 M NaOH in 95% ethanol solution. The cellulose fibers were regenerated 24 hours at room temperature. After completion of reaction time, the regenerated cellulose nanofibers were filtered through a Büchner filter, immersed in 500 mL dH2O for 1 minute and filtered again. This process was repeated until the conductivity of the final washing step was 0 μS/cm to make sure there were no residual NaOH, or acetate left. Finally, the nanofibers were dried in the oven over night at 60° C.

### Dividing of Cellulose Nanofibers

A 1% cellulose suspension was prepared by adding 5 g of regenerated cellulose to 500 ml of distilled water. The solution was added to the disperser and mixed for various timepoints at 18,000 rpm (IKA T25 digital Ultra Turrax). 100 mL samples labeled were removed after 1 (D1), 5 (D5), 15 (D15) and 60 (D60) minutes of mixing. The procedure was replicated with blender, but with the speed set to 7,000 rpm (LB20E Laboratory blender, Waring) (samples labeled: B1, B5, B15 and B60). After mixing, the fibers were dried in the oven over night at 60° C. The size distribution of the nanofibers was analysed by SEM and dynamic light scattering (DLS). SEM measurements and DLS measurements were performed as described in Example 2. The length of individual cellulose nanofibers was assessed from SEM images (4 induvial 10 μL drops for each timepoint). For the light scattering experiments, 10.000 fibers were measured per sample.

### Functionalization of Cellulose Nanofibers

Samples of regenerated cellulose fibers, blended or dispersed at various timepoints, were functionalized with quaternary ammonia (QA). Briefly, 1 g of regenerated cellulose from each time point was resuspended in 50 ml of 1.5 M NaOH solution and mercerised for 2 hours under stirring at room temperature. After the mercerization step, the temperature was increased to 80° C. and 2.5 ml CHPTAC was added dropwise. The reaction continued at 80° C. for 4 hours. After the reaction, the mixture was filtered through a Büchner filter, immersed in 200 mL dH2O for 1 minute and filtered again. This process was repeated until the conductivity of the final washing step was 0 μS/cm to make sure there were no residual CHPTAC or NaOH left. Finally, the filtered fibers were resuspended in water to a 2% solution and freeze dried for further experimentation.

### Results

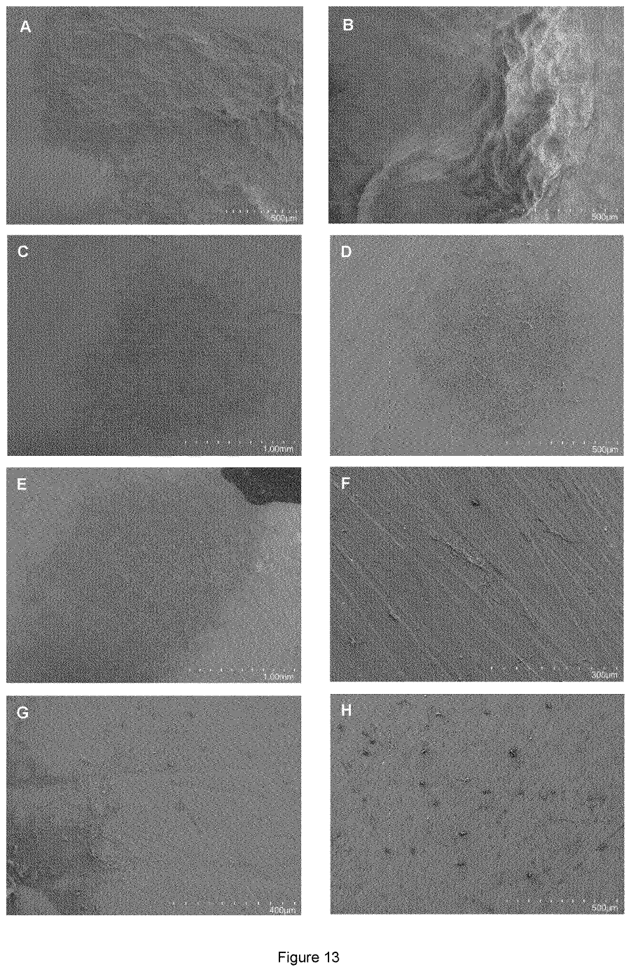

It is clear from the SEM images (FIG. 13A-H) that the disperser quickly provides a homogenous nanofiber population with relatively few long nanofibers and without any significant entanglement. While large chunks of uncut nanofibers are present in the samples after only 1 min of dividing (FIG. 13A-B), the disperser presents a finer population of nanofibers with only few smaller chunks of entangled nanofibers already after 5 min of dispersing (FIG. 13D). After 15 and 60 min of dispersing the presence of entangled nanofibers is almost completely eliminated (FIGS. 13F and 13H). In contrast, the blended samples comprise large chunks of entangled fibers even after 60 min of blending (FIG. 13G).

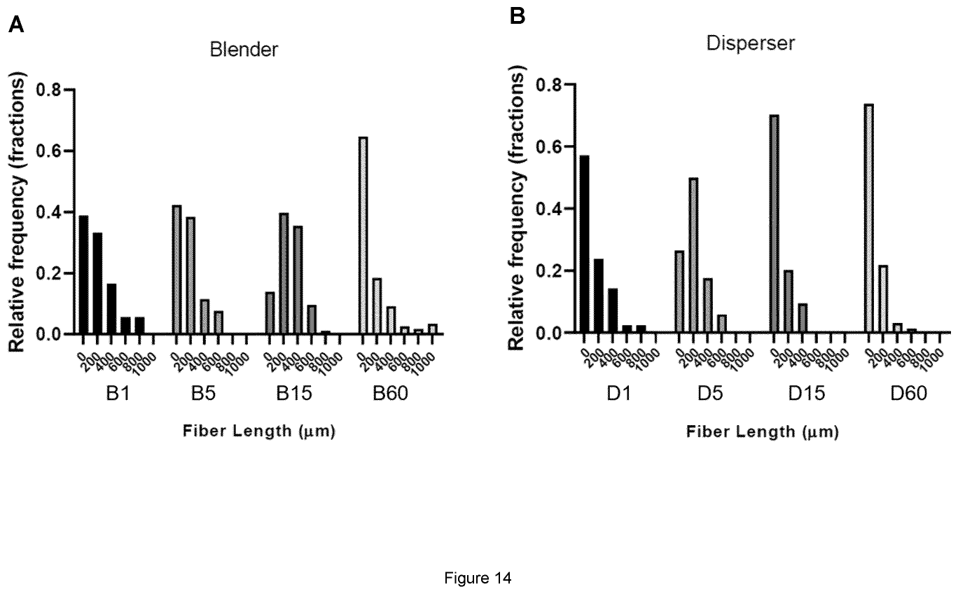

The data are summarised for each of the samples in FIG. 14A-B. It is clear that the frequency of long nanofibers is higher in the blended samples compared to the dispersed samples. Furthermore, there is a tendency towards longer processing times producing less long nanofibers.

This relative trend is supported by measurements of the nanofibers using light diffraction. These data are summarised in Table 3.

TABLE 3
Fiber length measurements of cellulose nanofibers prepared by
blending (B1, B5, B15, B60) and dispersing (D1, D5, D15, D60).
Measurements were performed on a Malvern Mastersizer S.

| | | | | D[4, 3] |
|---|---|---|---|---|
| | D[v, 0.1] | D[v, 0.5] | D[v, 0.9] | (μm) |
| | (μm) | (μm) | (μm) | Volume |
| | 10% below | 50% below | 90% below | weighted |
| Sample | this value | this value | this value | mean |
| B1 | 34 | 305 | 665 | 328 |
| B5 | 7 | 116 | 465 | 185 |
| B15 | 3 | 55 | 257 | 96 |
| B60 | 3 | 43 | 252 | 87 |
| D1 | 23 | 236 | 612 | 280 |
| D5 | 4 | 60 | 259 | 98 |
| D15 | 3 | 49 | 237 | 87 |
| D60 | 3 | 46 | 230 | 83 |

Overall it is desired that the mean length of the cellulose nanofibers is reduced. However it is also important that the population of nanofibers does not contain many long nanofibers as these may act as nucleus for larger clusters of nanofibers and entanglements. These clusters or entanglements of nanofibers are undesirable in the nanofibrous scaffold as large fractions of the surface area remains inaccessible to the cells seeded thereupon. Moreover, an inhomogeneous population of nanofibers comprising clusters and entanglements are undesirable because the variability between batches of the nanofibrous scaffold becomes inconsistent and therefore unreliable for cell growth.

### Conclusion

This example demonstrates that the means of dividing the cellulose nanofiber material influences the size distribution of the nanofibers, and in particular the fraction of longer fibers. Dispersing of the nanofibers is preferred because it rapidly removes longer fibers and yields the most homogenous population of cellulose nanofibers.

### Example 6: Nanofibrous Cellulose Scaffolds with Varying Amounts of Cellulose and Crosslinker

In this example the influence of cellulose concentration and the ratio of crosslinking agent to cellulose on the nanofibrous cellulose scaffold was assessed through scaffold integrity and cell growth. An overview of the samples prepared is given in Table 4.

TABLE 4
Cellulose concentration and crosslinking agent to cellulose ratio
used for preparation of nanofibrous cellulose scaffolds. The crosslinking
agent to cellulose ratio is given as both vol %/wt % and mol %/mol %.
The mol % of the cellulose material corresponds to per glucose unit.
Thus, the *10⁻³ mol %/mol % is a measure of the number of crosslinking
molecules per 1000 glucose units.

| | Crosslinking agent to cellulose ratio | |
|---|---|---|
| Cellulose | [vol %/wt %] | |
| concentration | [*10⁻³ mol %/mol %] | |
| [wt %] | First series | Second series |

| | | | | |
|---|---|---|---|---|
| 2.0 | 1:40 | | | |
| | 2.4 | | | |
| 1.0 | 1:20 | | | |
| | 4.7 | | | |
| 0.5 | 1:10 | | | |
| | 9.5 | | | |
| 0.25 | 1:5 | 1:25 | 1:5 | 1:1 |
| | 19.0 | 3.8 | 19.0 | 94.9 |
| 0.125 | 1:2.5 | 1:25 | 1:5 | 1:1 |
| | 37.9 | 3.8 | 19.0 | 94.9 |
| 0.06 | 1:1.25 | 1:25 | 1:5 | 1:1 |
| | 75.9 | 3.8 | 19.0 | 94.9 |
| 0.03 | 1.6:1 | | | |
| | 151.8 | | | |
| 0.015 | 3.2:1 | | | |
| | 303.6 | | | |

### Method

### Preparation of Nanofibrous Cellulose Scaffolds

A first series of nanofibrous cellulose scaffolds was prepared to find the optimal porosity range of the scaffold, i.e., large enough pores for the cells to penetrate the scaffold and have access to large surface area. Scaffolds were produced by crosslinking with a fixed amount of crosslinking agent to various cellulose concentrations. The procedure as described in Example 1 was followed with the following changes to concentrations.

Nanofibrous cellulose scaffolds with different porosities were prepared by crosslinking various concentrations of QA functionalized cellulose (2.0, 1.0, 0.5, 0.25, 0.125, 0.06, 0.03 and 0.015% cellulose). 0.5 μl crosslinking agent (Kymene)/ml cellulose suspension was added to each concentration of functionalized cellulose. 500 μl of suspension mixed with crosslinking agent was subsequently added to a 48-well mold (maximum fill volume 0.5 ml, diameter 12 mm, height 10 mm). The molds were frozen to −80° C. and thereafter freeze-dried. Finally, the scaffolds were cured in the oven at 120° C. for 3 hours on an aluminium tray.

A second series of nanofibrous cellulose scaffolds was prepared to evaluate the crosslinking agent to cellulose ratio for a subset of cellulose concentrations (0.06%, 0.125% and 0.25%) that were deemed beneficial. The amount of crosslinking agent (Kymene) added to the cellulose suspensions was adjusted so that samples with either low (3.8*10⁻³ mol %/mol %), medium (19.0*10⁻³ mol %/mol %), or high (94.9*10⁻³ mol %/mol %) crosslinking agent to cellulose ratios were prepared (see Table 4). The low, medium and high samples corresponded to addition of 0.04, 0.2, and 1 μl crosslinking agent pr. mg. cellulose material, respectively.

### Cell Growth on Nanofibrous Cellulose Scaffold

Cell growth experiment on nanofibrous cellulose scaffolds was performed in 24 well plate format and using HEK293T cells (ATCC CTRL-3216) at passage 8 (92% viability on the day of inoculum with scaffolds) for the first series or at passage 10 (97.5% viability on the day of inoculum with scaffolds) for the second series.

The scaffolds were washed 3 times in DPBS. One cellulose scaffold was placed in each well on a 24-well plate with 1 ml of cell culture media (DMEM with high glucose and GlutaMAX Supplement and pyruvate, 10% heat-inactivated FBS and 1% penicillin-streptomycin), and 300000 cells were seeded on each scaffold and left on an orbital shaker (16 mm orbit) at 35 rpm overnight in an incubator (at 37° C. with 5% CO₂).

The next morning, each well was topped up with 0.5 ml media to reach final volume of 1.5 ml per well. The rpm of the orbital shaker (16 mm orbit) was increased to 65 rpm. 24, 48 and 72 hours after seeding, three replicate scaffolds were moved with a tweezer into 15 ml tubes and enzymatic dissociation of cells was used in combination with the microcarrier program of the NucleoCounter (NC-202) to assess the cell density. The mean values obtained, corresponding to total cell concentration/scaffold (in one well), were determined and captured as histograms.

Fluorescence microscopy of DAPI-stained and optically cleared samples was performed at 72 hours using a confocal microscope.

### Results

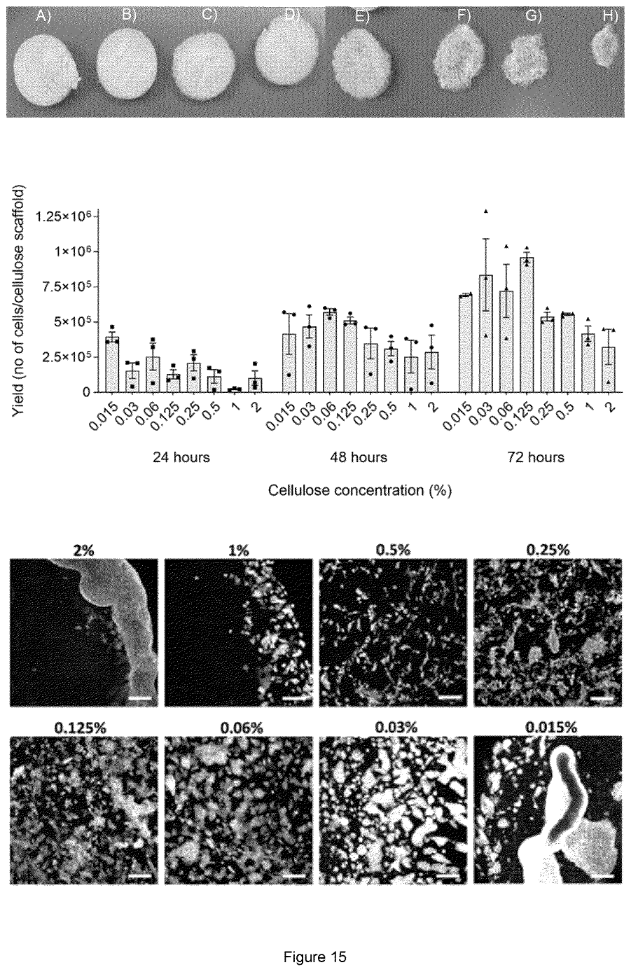

Nanofibrous cellulose scaffolds prepared from cellulose concentrations ranging from 0.015% to 2% were prepared (FIG. 15, top). There was a clear tendency towards scaffolds of lower cellulose concentration (higher porosity) loosing shape.

Cell growth was particularly enhanced on scaffolds prepared from cellulose concentrations of 0.03% to 0.125% (FIG. 15, middle). Especially, the scaffold prepared from a cellulose concentration of 0.125% displayed increased growth and low variability between replicates. Both the 0.015% and 0.03% samples were challenging to handle due to the lightweight nature and fragility, leading to a propensity to break. The samples prepared from 1% and 2% cellulose concentrations displayed a denser structure that appear to predominantly support cell growth on the outer surfaces as penetration into the scaffold is hindered due to small pore size. This observation was corroborated by cell-staining (FIG. 15, bottom) which confirmed the observation that that limited cell penetration occurred for the 1% and 2% samples.

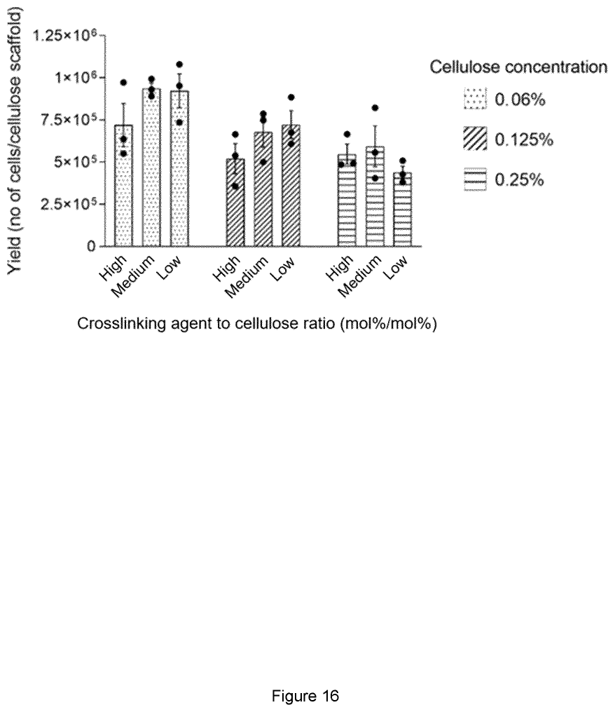

For the nanofibrous cellulose scaffolds with varying amounts of crosslinking agent the scaffolds prepared from 0.06% and 0.125% cellulose concentrations enabled higher cell growth than the scaffold prepared from 0.25% cellulose concentration (FIG. 16). This confirmed the previous findings (FIG. 15, middle). Without being bound by theory, this difference may be due to the higher porosity of the scaffold prepared from lower cellulose concentrations. The data also show that crosslinking agent to cellulose ratios below 94.9*10⁻³ mol %/mol % leads to enhanced cell growth, e.g. for the scaffold of higher porosity.

### Conclusion

This example demonstrates that nanofibrous cellulose scaffolds can be prepared from a range of cellulose concentrations. The cellulose concentration alters the porosity of the scaffold and cellulose concentrations in the range of 0.03% to 0.125% are preferred for their properties that support cell proliferation by facilitating increased cell entry into the pores. Furthermore, it is advantageous to crosslink the cellulose nanofibers with crosslinking agent to cellulose ratios of less than 94.9*10⁻³ mol %/mol %.

### Example 7: Mechanical Stability of Nanofibrous Cellulose Scaffold

In this example the mechanical stability of the nanofibrous cellulose scaffold was probed with the aim of evaluating its suitability in an industrial setup including shear stress from agitation.

### Method

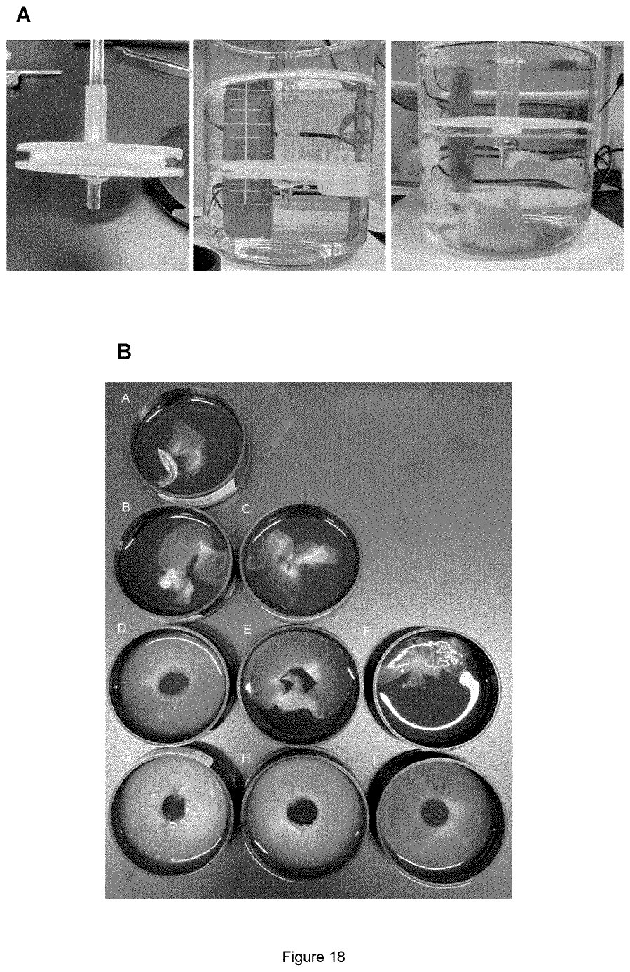

Discs of nanofibrous cellulose scaffold were produced in a larger format with a diameter of 57 mm to fit a bioreactor compared to the scaffolds produced in Example 6 (smaller diameter of 12 mm). The stability was tested based on the ability of the discs to be removed from the mold without breaking and the ability of the discs to withstand the shear stress applied when stirred in a mockup reactor (FIG. 18A).

Discs of nanofibrous cellulose scaffold with different porosities were prepared by crosslinking various concentrations of QA functionalized cellulose (0.25%, 0.125%, 0.06% and 0.03%) as described in Example 6. 0.2 μl crosslinking agent per mg cellulose (19.0*10⁻³ mol %/mol %) was added to each concentration of functionalized cellulose. Different thicknesses of the scaffolds were obtained by filling the molds with different volumes (3, 10, 17 and 25 ml) of cellulose suspension. The molds had an inner diameter of 57 mm, a maximum fill height of 10 mm and a total volume of 25 ml. After filling the molds with different volumes, they were frozen at −80° C. and thereafter freeze dried. Finally, the scaffolds were cured in the oven at 120° C. for 3 hours on an aluminium tray.

The mechanical stability for each scaffold was tested in a mockup bioreactor. Each scaffold was placed between two perforated support discs attached to a stirring rod (FIG. 18A, left). The stirring rod was attached to a Nano Star 7.5 Digital stirrer (IKA) and immersed into a beaker with 500 ml of PBS to mimic the rotating motion of a stirred bioreactor (FIG. 18A, middle). The stirring speed was set to 50 rpm and stability was observed over time. FIG. 18A (right) illustrates a broken scaffold (sample IIB) due to the shear forces applied during rotation.

### Results

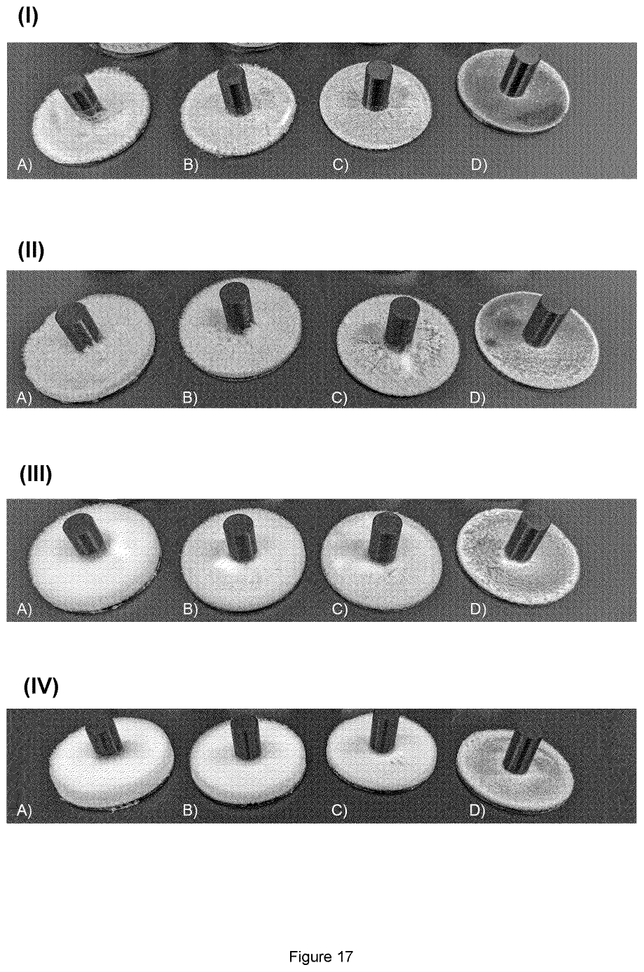

The methodology led to the preparation of a wide range of nanofibrous cellulose scaffold with different thicknesses and densities/porosities (FIG. 17(I)-(IV)). From visual inspection it was clear that the scaffolds prepared from the highest cellulose concentrations, e.g. 0.25% (FIG. 17(IV)) and 0.125% (FIG. 17(III)), produced the most coherent discs. An overview of the measured thickness of the scaffolds is presented in table 5.

TABLE 5
Samples prepared for testing of mechanical stability.

| | Volume suspension | Cellulose concentration | Thickness |
|---|---|---|---|
| Sample | (ml) | (wt %) | (mm) |
| IA | 25 | 0.03 | 5 |
| IB | 17 | 0.03 | 3 |
| IC | 10 | 0.03 | 1 |
| ID | 3 | 0.03 | 0.5 |
| IIA | 25 | 0.06 | 7 |
| IIB | 17 | 0.06 | 5 |
| IIC | 10 | 0.06 | 3 |
| IID | 3 | 0.06 | 0.5 |
| IIIA | 25 | 0.125 | 9 |
| IIIB | 17 | 0.125 | 6 |
| IIIC | 10 | 0.125 | 3 |
| IIID | 3 | 0.125 | 0.5 |
| IVA | 25 | 0.25 | 9 |
| IVB | 17 | 0.25 | 6 |
| IVC | 10 | 0.25 | 3 |
| IVD | 3 | 0.25 | 1 |

There was a tendency for scaffolds prepared from less than 0.125% cellulose concentration to collapse upon freeze-drying leading to decreased thickness compared to the more dense scaffolds. It was not possible to remove the thinnest scaffolds made from a volume suspension of only 3 ml from the mold without breakage. At higher suspension volumes it was easier to handle the resulting thicker scaffolds.

The test in the mockup bioreactor revealed that only scaffolds prepared from cellulose concentrations of 0.013% and (17 ml (partly), 25 ml) and 0.25% (10 ml, 17 ml, and 25 ml) were capable of withstanding the shear stress of the mockup bioreactor.

### Conclusion

This example demonstrates that it is possible to produce nanofibrous cellulose scaffolds with sufficient mechanical strength to resist breakage when used as microcarrier in a stirred bioreactor. The scaffolds are preferably prepared from a cellulose concentration of at least 0.125%, and may advantageously have a thickness in the range of 6 mm to 10 mm. More dense scaffolds may be prepared at a thickness of 3 mm and still preserve structural integrity in a stirred bioreactor.

### Example 8: Cell Growth on Nanofibrous Cellulose Scaffold in Commercial Bioreactor

In this example the nanofibrous cellulose scaffold is tested in a commercial bioreactor to evaluate its suitability as microcarrier.

### Method

Pilot nanofibrous cellulose scaffolds were prepared in disc-form as described in Example 7 by crosslinking a 1% suspension of QA functionalized cellulose by addition of 1.0 μl crosslinking agent per ml cellulose suspension. 10 ml of the suspension was added to the molds with an inner dimension of 57 mm and maximum fill height of 10 mm. The molds were frozen at −80° C. and thereafter freeze dried. Finally, the scaffolds were cured in the oven at 120° C. for 3 hours on an aluminium tray. The thickness of the scaffolds was around 3 mm, and the weight of each disc was 100 mg.

Three discs made from the nanofibrous cellulose scaffolds corresponding to approximately 1 m² surface area, were mounted in 1 L CellBRX bioreactor whereafter 300,000,000 HEK293 cells were inoculated into the bioreactor for them to adhere onto the scaffolds. Cells were grown for 5 days and cell count and glucose consumption was probed every day.

### Results

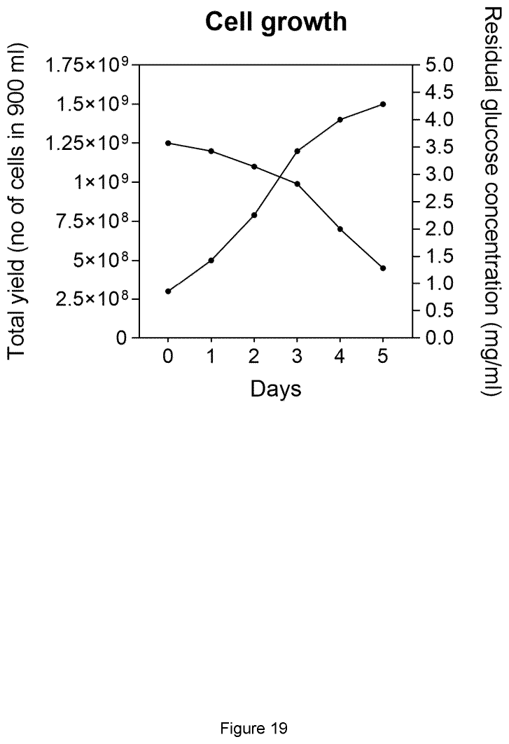

Cells attached and grew on the nanofibrous cellulose scaffolds discs in the 1 L CellBRX bioreactor system (FIG. 19)) as shown by increased cell density over time and concurrent glucose consumption.

### CONCLUSION

This example demonstrates that the nanofibrous cellulose scaffolds are suitable for use in bioreactors in an industrial setup for culturing large populations of cells.

1. A method of preparing a nanofibrous cellulose scaffold, said method comprising:
dividing an initial cellulose nanofiber material into a processed cellulose nanofiber material,
crosslinking the processed cellulose nanofiber material, and
drying the processed cellulose nanofiber material,
wherein said dividing is achieved by dispersing,
thereby providing the nanofibrous cellulose scaffold.

2-22. (canceled)

23. The method according to claim 1, wherein the said dispersing is performed with a high-speed disperser.

24. The method according to claim 1, wherein dispersing is performed for at least 2 min.

25. The method according to claim 1, wherein the mean length of the cellulose nanofibers in said processed cellulose nanofiber material is in the range of about 70 μm to about 120 μm.

26. The method according to claim 1, wherein the processed cellulose nanofiber material is crosslinked by addition of a crosslinking agent selected from the group consisting of epichlorohydrin, formaldehyde dimethyl acetate (FDA), maleic anhydride (MAL), 3-glycidoxypropyltrimethoxysilane (GPS), citric acid, and 1,2,3,4-butanetetracarboxylic acid.

27. The method according to claim 26, wherein the crosslinking agent is an epichlorohydrin.

28. The method according to claim 1, wherein the ratio of crosslinking agent to processed cellulose nanofiber material is in the range of about 15*10⁻³ mol %/mol % to about 80*10⁻³ mol %/mol %.

29. The method according to claim 1, wherein the concentration of cellulose nanofibers in the processed cellulose nanofiber material is about 0.01 wt % to about 1 wt %.

30. The method according to claim 1, wherein the initial cellulose nanofiber material is prepared by electrospinning, meltblowing, drawing, self-assembly, template synthesis, or thermal-induced phase separation.

31. The method according to claim 1, wherein the drying comprises freezing or lyophilization of the processed cellulose nanofiber material.

32. The method according to claim 1, wherein the drying is followed by curing of the dried processed cellulose nanofiber material.

33. The method according to claim 1 further comprising functionalizing the processed cellulose nanofiber strands by addition of a reagent comprising a functional moiety, wherein the functionalizing of the processed cellulose nanofiber strands precedes or follows the crosslinking.

34. A nanofibrous cellulose scaffold obtainable from the method according to claim 1.

35. A nanofibrous cellulose scaffold comprising a processed cellulose nanofiber material with a mean length of the cellulose nanofibers of less than about 250 μm, and wherein the processed cellulose nanofiber material comprises crosslinks induced by a crosslinking agent.

36. The nanofibrous cellulose scaffold according to claim 35, wherein the ratio of crosslinking agent to processed cellulose nanofiber material is in the range of about 15*10⁻³ mol %/mol % to about 80*10⁻³ mol %/mol %.

37. The nanofibrous cellulose scaffold according to claim 35, wherein the processed cellulose nanofiber material is functionalized with a functional moiety.

38. The nanofibrous cellulose scaffold according to claim 35, wherein the nanofibrous cellulose scaffold is provided as a dry continuous material.

39. The nanofibrous cellulose scaffold according to claim 35, wherein the nanofibrous cellulose scaffold is a disc with a thickness in the range of about 3 mm to about 30 mm.

40. The nanofibrous cellulose scaffold according to claim 35, wherein the nanofibrous cellulose scaffold has a BET surface area of at least about 40000 cm²/g.

41. A microcarrier comprising the nanofibrous cellulose scaffold according to claim 35.

42. A cell culturing device comprising a container loaded with the nanofibrous cellulose scaffold according to claim 35.

43. A method for cell culturing comprising:
(i) providing the cell culturing device according to claim 42,
(ii) adding a composition comprising a cell population to the cell culturing device,
(iii) incubating the cell population to provide a proliferated cell population, and
(iv) optionally, extracting the proliferated cell population from the cell culturing device.
